(19) United States
(12) Patent Application Publication
(10) Pub. No.: US 2022/0296683 A1
(43) Pub. Date: Sep. 22, 2022

(54) ORAL DELIVERY SYSTEM

(71) Applicant: University College Dublin, 4 Dublin (IE)

(72) Inventors: David James Brayden, Belfield, Dublin 4 (IE); Delyan HRISTOV, Belfield, Dublin 4 (IE); Eugene MAHON, Belfield, Dublin 4 (IE); Sourav BHATTACHARJEE, Belfield, Dublin 4 (IE); Jason BEIRNE, Belfield, Dublin 4 (IE); Fiona MCCARTNEY, Belfield, Dublin 4 (IE)

(73) Assignee: University College Dublin, 4 Dublin (IE)

(21) Appl. No.: 17/762,225

(22) PCT Filed: Sep. 21, 2020
(86) PCT No.: PCT/EP2020/076339

§ 371 Date: Mar. 21, 2022

(30) Foreign Application Priority Data
Sep. 20, 2019 (GB) 1913606.8
Sep. 20, 2019 (GB) 1913609.2

(51) Int. Cl. A61K 38/28; A61K 9/16; A61K 38/26; A61P 3/10

(52) U.S. Cl. CPC A61K 38/28; A61K 9/1641; A61K 9/1611; A61K 38/26; A61K 9/1694; A61P 3/10

(57) ABSTRACT

The invention provides a method of preparing a silica-protein sub-micron particle, the method comprising contacting, in an aqueous medium having a pH in the range of about 7.5 to 10 and having an ionic strength in the range of about 1.2 to 60 mM, (i) protein sub-micron particle cores comprising 0.2 to 3 μmoles, per mg of the protein sub-micron particle cores, of a basic compound of formula (I) or an ester and/or amide thereof wherein n is an integer selected from 1 to 10, and p is an integer selected from 1 to 3, with (ii) orthosilicic acid or an ester thereof, in an amount equivalent to 5 to 60% (w/w) orthosilicic acid per mg of the protein sub-micron particle cores, to form the silica-protein sub-micron particle. The invention also provides a method of making a GLP-1 receptor agonist sub-micron particle core, by a. contacting a peptide in an aqueous medium at a pH of about 5.0-8.0 and comprising about 0.3-1.3 mM peptide, wherein the peptide is a GLP-1 receptor agonist, with the above-mentioned basic compound of formula (I) or an ester and/or amide thereof: wherein n is an integer selected from 1 to 10, and p is an integer selected from 1 to 3; in a molar ratio between the peptide and the basic compound of from 1:about 20 to 1:about 90 to form a step a. reaction mixture having a pH of about 7.5 to 10.0; optionally about 9.5; b. contacting the step a. reaction mixture with zinc to form a step b. reaction mixture, wherein the ratio of peptide:basic compound:zinc in the step b. reaction mixture is 1:about 20 to 110:9 to 30; whereby the GLP-1 receptor agonist sub-micron particle core is formed. The invention also provides a method of making an insulin sub-micron particle core, the method comprising the steps of: a. contacting insulin in an aqueous medium having a pH of less than about 6.0 and comprising about 0.50 to about 1.50 mg/mL insulin, with the aforementioned basic compound of formula (I) or an ester and/or amide thereof; in a molar ratio between the insulin and the basic compound of from about 1:40 to about 1:140 to form a step a. reaction mixture having a pH of about 7.5 to 10; b. contacting the step a. reaction mixture with zinc to form a step b. reaction mixture, wherein the molar ratio of insulin:zinc in the step b. reaction mixture is 1:about 0.5 to about 10; whereby the insulin sub-micron particle core is formed.

Nanoparticle constructs for oral protein delivery must protect and present the payload at the small intestinal epithelium. In a reproducible, scalable, surfactant-free process, a nano-sized core was formed with a model peptide (insulin), in ratios with zinc chloride and L-arginine, which was then cross-linked with silica to form an outer shell. The nanoparticles (SiNP) entrapped insulin in high concentration, preserved its structure, and released it at pH values present in the small intestine. The SiNP delivered insulin to the circulation and reduced plasma glucose in a rat jejunal instillation model. The delivery mechanism required residual L-arginine to act as a permeation enhancer for SiNP-released insulin in the jejunum. The process could be varied in terms of ratios of the core components for entrapping other payloads including the glucagon-like Peptide 1 analogue, exenatide, and bovine serum albumen. The process is the basis of a platform for oral peptide and protein delivery.

Introduction

The number of approved biopharmaceuticals has steadily increased, most of which are injectables¹. Oral delivery is the preferred route for biopharmaceuticals due to convenience compared to injections². Further rationale arises for the estimated 5% of patients who suffer from needle phobia³, whereas others undergo painful injections for specific conditions using low gauge needles⁴. For insulin and glucagon-like Peptide 1 (GLP-1) analogue treatments for Type II diabetes patients, the rationale to develop oral formats to promote earlier adoption has been advocated in order to achieve better outcomes for patients who are typically reluctant to endure injections before they have to⁵. Furthermore, the oral route for insulin would mimic the physiological route of delivery to the liver⁶, would protect β cells of the pancreas, and can potentially reduce side-effects from sub-cutaneous (s.c.) injections, from which only a relatively small proportion of insulin reaches the liver⁷. Nonetheless, since insulin is a low therapeutic index molecule, low and variable oral bioavailability seen in clinical trials for oral insulin formulations would likely carry risk for patients⁸. In the current study, insulin was therefore selected as a model peptide. Clinical development of an oral Glucagon-1-like Peptide 1 (GLP-1) analogue is however more advanced, with a daily oral version of semaglutide recently having completed Phase III trials⁹.

High molecular weight, hydrophilicity, lability, and low intestinal permeability mitigate against delivering peptides orally¹⁰. The formulations in clinical trials therefore mostly comprise intestinal permeation enhancers (PEs) and peptidase inhibitors^{11, 12}. Nonetheless, oral peptide bioavailability for such technologies is only ˜1-2% relative to s.c. administration^{13, 14}. Even if efficacy is obtained, there will likely be safety questions relating to intestinal damage, microbiome changes, and association with coeliac and inflammatory bowel diseases in respect of chronic dosing of high doses of enhancers¹⁵. As an alternative to use of high doses of enhancers, nanotechnologies can both protect peptides during gastrointestinal (GI) tract transit and, if suitably coated with hydrophilic materials, can enable mucus permeation^{16, 17}. There is considerable debate over whether peptide-loaded nanoparticles are actually taken up by intestinal epithelia to an extent that would account for peptide levels in plasma in vivo. Publications from the EU H2020 consortium, TRANS-INT, tested numerous prototypes, leading to an overall conclusion that in vivo epithelial nanoparticle uptake was low, with most appearing to be either stuck in intestinal mucus, with some associating with the epithelium, e.g.¹⁸. Several of these constructs¹⁹⁻²¹, and indeed two of the original pioneering nanoparticle oral peptide compositions^{22, 23}, comprised excipients known to act as permeation enhancers. A reasonable interpretation therefore is that excipients used as components of the majority of nanoparticle constructs in the literature (with the exception of nanoparticles made with receptor-targeting ligands^{24, 25}), promote uptake of particle-released peptide in the small intestine.

Our hypothesis therefore was that an oral peptide nanoparticle should comprise a simple synthetic scalable process with potential for high loading. In addition, the particle should release its payload in high concentration close to the epithelium, triggered by the higher pH of the small intestine. Intestinal epithelial permeability should be assisted by co-formulated excipients. The components we selected were substances with a history of use in humans in order to alleviate toxicological risk. Silica is attractive as it is biocompatible, FDA-approved, inert, can be functionalized, and has a history of use in large nanoparticle formats in biomedical, food, and food packaging applications²⁶. Moreover, it has already been used in oral peptide nanoparticle constructs either as a core, over which excipients and peptides are attached²⁷, as a core with peptide followed by coating with mucoadhesive²⁸-, muco-permeating-²⁹, and enteric coating polymers³⁰, and also as a mesoporous structure to contain embedded peptides³¹⁻³³. It can respond to pH changes, so that it is intact in the stomach but releases in the small intestine³⁴. Here, we present a structure in which payloads including human insulin and the GLP-1 analogue, exenatide³⁵, were self-assembled as a core in the presence of the excipients, L-arginine (L-arg), and zinc chloride (ZnCl₂). While L-arg was included primarily in order to aid insulin stabilisation³⁶, we noted that L-arg (but not ZnCl₂) is an established intestinal permeation enhancer for insulin³⁷. The core particle was then crosslinked with silica to form silica-coated nanoparticles (SiNPs). The resulting SiNPs fulfilled target physicochemical and bioactivity criteria, while batches could be synthesised reproducibly with predictable characteristics. Importantly, the mechanism of achieving insulin delivery across the intestine was based on L-Arg released from the particle enabling permeation of the released insulin in the jejunum.

According to a first aspect of the invention, there is provided a method of preparing a silica-protein sub-micron particle, the method comprising contacting, in an aqueous medium having a pH in the range of about 7.5 to 10 and having an ionic strength in the range of about 1.2 to 60 mM,
(i) protein sub-micron particle cores comprising 0.2 to 3 μmoles, per mg of the protein sub-micron particle cores, of a basic compound of formula (I) or an ester and/or amide thereof

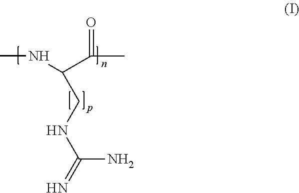

wherein n is an integer selected from 1 to 10, and p is an integer selected from 1 to 3,
with
(ii) orthosilicic acid or an ester thereof, in an amount equivalent to 5 to 60% (w/w) orthosilicic acid per mg of the protein sub-micron particle cores, to form the silica-protein sub-micron particle.

Optionally, the protein sub-micron particle cores comprising the basic compound of formula (I) are formed by
(a) dispersing 0.5 to 5.5 mg/mL of the protein in an aqueous medium having a pH of about 7.5 to 10 and having an ionic strength in the range of about 1.2 to 60 mM; and
(b) contacting the product of step (a) with the basic compound of formula (I) under reaction conditions to form the protein sub-micron particles comprising the basic compound of formula (I) in an amount equivalent to 0.2 to 2 mmoles of the basic compound of formula (I) per mg of sub-micron particles.

Optionally, the protein sub-micron particle cores comprising the basic compound of formula (I) are formed by
(c) dispersing 0.5 to 5.5 mg/mL of the protein and the basic compound of formula (I) in an aqueous medium having a pH of about 7.5 to 10 and having an ionic strength in the range of about 1.2 to 60 mM to form the protein sub-micron particle comprising the basic compound of formula (I) in an amount equivalent to 0.2 to 2 mmoles of arginine per mg of sub-micron particles.

Optionally, the basic compound of formula (I) is L or D arginine, or an ester and/or amide thereof.

Optionally, the basic compound of formula (I) is a poly arginine (L or D), or an ester and/or amide thereof.

Optionally, the poly arginine is a dimer or a trimer.

Optionally, the orthosilicic acid or the ester thereof is orthosilicic acid.

Optionally, the orthosilicic acid or the ester thereof is an orthosilicate ester.

Further optionally, the silicate ester retains 0 to 3 hydroxyl groups of the 4 hydroxyl groups of orthosilicic acid; or each hydroxyl group of orthosilicic acid is independently esterified with C₁₋₅ alkyl; optionally, ethyl.

Optionally, each hydroxyl group is esterified with ethyl.

Optionally, the orthosilicic acid or the ester thereof, is present in an amount equivalent to about 10 to 50% (w/w) orthosilicic acid per mg of protein sub-micron particles; wherein, optionally, when the protein is insulin, about 10 to 20% (w/w) orthosilicic acid per mg of protein sub-micron particles is present; and, when the protein is exenatide, about 10 to 50% (w/w) orthosilicic acid per mg of protein sub-micron particles is present.

Optionally, in step (a), about 0.85 to 4.00 mg/mL of the protein is dispersed; wherein, optionally, when the protein is insulin, about 0.85 to 1.5 mg/mL of the protein is dispersed; and, when the protein is exenatide, about 1.0 to 4.00 mg/mL of the protein is dispersed.

Optionally, in step (c), about 0.85 to 4.00 mg/mL of the protein is dispersed; wherein, optionally, when the protein is insulin, about 0.85 to 1.5 mg/mL of the protein is dispersed; and, when the protein is exenatide, about 1.0 to 4.0 mg/mL of the protein is dispersed.

Optionally, the pH of the aqueous medium is in the range of about 8.9 to 9.7.

Optionally, the ionic strength of the aqueous medium is in the range of about 2 to 35 mM.

Optionally, the contacting step is carried out a temperature in the range of about 18 to 40° C.

Optionally, the method further comprises the step of separating the silica-protein sub-micron particle from the aqueous medium by centrifugation.

Optionally, the silica-protein sub-micron particle has a diameter of between about 30 and 600 nm; optionally between about 50 and 300 nm; further optionally between about 80 and 300 nm.

Optionally, the protein is insulin and the silica-protein sub-micron particle has a diameter of between about 80 and 300 nm; or the protein is exenatide and the silica-protein sub-micron particle has a diameter of between about 30 and 600 nm.

According to a further aspect of the invention, there is provided a method of making a GLP-1 receptor agonist sub-micron particle core, the method comprising the steps of:

a. contacting a peptide in an aqueous medium at a pH of about 5.0-8.0 and comprising about 0.3-1.3 mM peptide, wherein the peptide is a GLP-1 receptor agonist,

with

a basic compound of formula (I) or an ester and/or amide thereof:

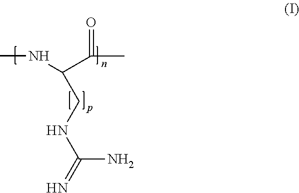

wherein n is an integer selected from 1 to 10, and p is an integer selected from 1 to 3;

in a molar ratio between the peptide and the basic compound of from 1:about 20 to 1:about 90 to form a step a. reaction mixture having a pH of about 7.5 to 10.0; optionally about 9.5;

b. contacting the step a. reaction mixture with zinc to form a step b. reaction mixture, wherein the ratio of peptide:basic compound:zinc in the step b. reaction mixture is 1:about 20 to 110:9 to 30, optionally between 1:48:16 to 22 and 1:90:20 to 25;

whereby the GLP-1 receptor agonist sub-micron particle core is formed.

Optionally, the GLP-1 receptor agonist is selected from exendin-4, exenatide (Byetta, Bydureon), liraglutide (Victoza), lixisenatide (Lyxumia), dulaglutide (Trulicity) albiglutide (Tanzeum) sitagliptin (Januvia, Janumet, Janumet XR, Juvisync), saxagliptin (Onglyza, Kombiglyze XR), alogliptin (Nesina, Kazano, Oseni), semaglutide (Ozempic) and linagliptin (Tradjenta, Jentadueto); optionally is exenatide or analogues thereof.

Further optionally, the basic compound is arginine and the peptide is exenatide; and the concentration, in the step b. reaction mixture, of Zn is between about 6.5 and about 8.5 mM when the arginine is between about 25 and about 55 mM.

Optionally, the amount of GLP-1 receptor agonist, in the protein sub-micron particle core, is between about 20% w/w and about 80% w/w; optionally between about 30% w/w and about 70% w/w; further optionally between about 35% w/w and about 60% w/w of the protein sub-micron particle core.

According to a further aspect of the invention, there is provided a method of making an insulin sub-micron particle core, the method comprising the steps of:

a. contacting insulin in an aqueous medium having a pH of less than about 6.0 and comprising about 0.50 to about 1.50 mg/mL insulin,

with

a basic compound of formula (I) or an ester and/or amide thereof:

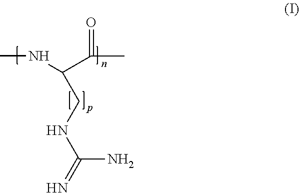

wherein n is an integer selected from 1 to 10, and p is an integer selected from 1 to 3; in a molar ratio between the insulin and the basic compound of from about 1:40 to about 1:140 to form a step a. reaction mixture having a pH of about 7.5 to 10, optionally about 8.0 to 10.0;

b. contacting the step a. reaction mixture with zinc to form a step b. reaction mixture, wherein the molar ratio of insulin:zinc in the step b. reaction mixture is 1:about 0.5 to about 10;

wherein, optionally, the molar ratio of insulin:basic compound:zinc in the step b. reaction mixture is 1:about 66 to about 100:about 3 to about 4.5;

whereby the insulin sub-micron particle core is formed.

Optionally, the insulin in the aqueous medium comprises about 0.9-about 1.2 mg/mL insulin, optionally about 1 mg/mL insulin.

Optionally, wherein n is 1 and p is 3 whereby the basic compound is arginine; and

the concentration, in the step b. reaction mixture, of Zn is about 0.35-0.65 mM, optionally about 0.5 mM, when the arginine concentration is 2 mg/mL; or

the concentration, in the step b. reaction mixture, of Zn is about 0.6-0.9 mM, optionally about 0.75 mM, when the arginine concentration is about 3 mg/mL.

Optionally, the amount of insulin, in the protein sub-micron particle core, is between about 35% w/w and about 95% w/w; optionally between about 45% w/w and about 90% w/w; further optionally between about 50% w/w and about 75% w/w of the protein sub-micron particle core.

Optionally, the insulin is a human insulin or an analog thereof; optionally selected from Lispro (Eli Lilly), Aspart (Novo Nordisk), Glulisine (Sanofi-Aventis), Detemir insulin (Novo Nordisk), Degludec insulin (Novo Nordisk), Glargine insulin (Sanofi-Aventis).

Optionally, the aqueous medium has a pH of less than about 4.0, optionally about pH 2.5.

Optionally, the basic compound is L-arginine.

Optionally, the core comprises the peptide, the basic compound and the zinc; or the core consists of the peptide, the basic compound and the zinc.

Optionally, the method further comprises contacting, in an aqueous medium having a pH in the range of about 7.5 to 10 and having an ionic strength in the range of about 1.2 to 60 mM,
(i) protein sub-micron particle cores comprising 0.2 to 3 μmoles, per mg of the protein sub-micron particle cores, of a basic compound of formula (I) or an ester and/or amide thereof

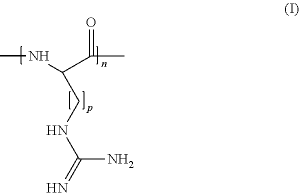

wherein n is an integer selected from 1 to 10, and p is an integer selected from 1 to 3,
with
(ii) orthosilicic acid or an ester thereof, in an amount equivalent to 5 to 60% (w/w) orthosilicic acid per mg of the protein sub-micron particle cores, to form the silica-protein sub-micron particle.

Alternatively, the aforementioned protein sub-micron particle cores are enterically coated in any suitable manner.

As used herein, the term “enteric” refers to a coating that permits transition of the protein sub-micron particle cores through the stomach to the small intestine before the protein is released therefrom. Examples of enteric coating polymers include hydroxypropylmethylcellulose phthalate (HPMCP), hydroxypropylmethylcellulose acetate succinate (HPMCAS) and RL100, and HP-55, which can be coated on the protein sub-micron particle cores and dissolved in the upper intestinal conditions. Other examples include pH-sensitive copolymers of poly(methacrylic acid-co-N-vinyl caprolactam) for the pH-sensitive oral delivery of active ingredients such as insulin and pH-sensitive biodegradable copolymers such as EUDR AGIT® (Evonik) acr ylic drug delivery polymers as an enteric coating polymer that releasesthe protein (such as insulin) only at neutral pH, thereby preventing its degradation at acidic pH.

Optionally, the protein sub-micron particle cores comprising the basic compound of formula (I) are formed by
(a) dispersing 0.5 to 5.5 mg/mL of the protein in an aqueous medium having a pH of about 7.5 to 10 and having an ionic strength in the range of about 1.2 to 60 mM; and
(b) contacting the product of step (a) with the basic compound of formula (I) under reaction conditions to form the protein sub-micron particles comprising the basic compound of formula (I) in an amount equivalent to 0.2 to 2 mmoles of the basic compound of formula (I) per mg of sub-micron particles.

Optionally, the protein sub-micron particle cores comprising the basic compound of formula (I) are formed by

(c) dispersing 0.5 to 5.5 mg/mL of the protein and the basic compound of formula (I) in an aqueous medium having a pH of about 7.5 to 10 and having an ionic strength in the range of about 1.2 to 60 mM to form the protein sub-micron particle comprising the basic compound of formula (I) in an amount equivalent to 0.2 to 2 mmoles of arginine per mg of sub-micron particles.

The invention also provides a silica-protein sub-micron particle formed by the aforementioned methods, for use in the treatment of diabetes. Optionally, the protein is insulin and the diabetes is Type I diabetes. Further optionally, the diabetes is Type II diabetes.

In the drawings and tables,

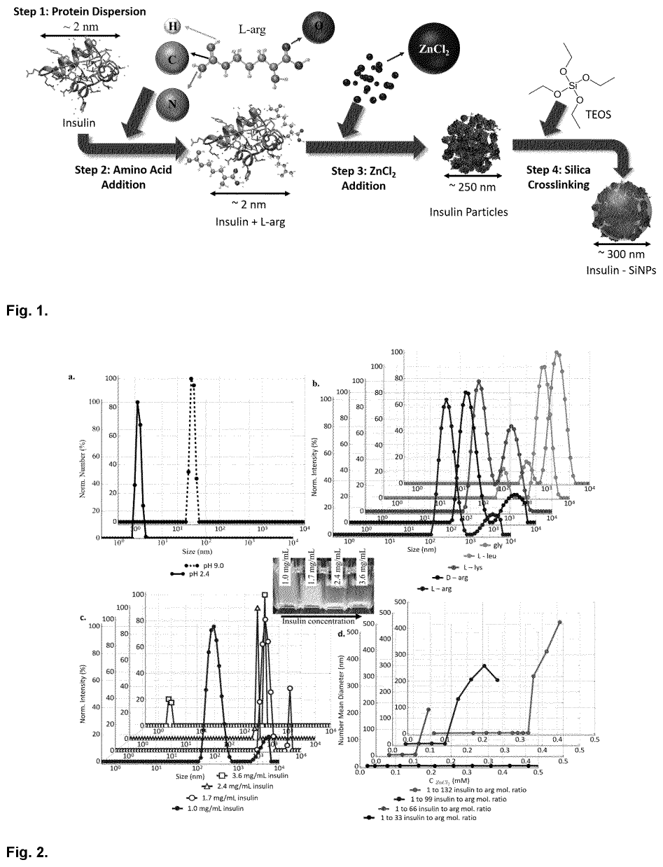

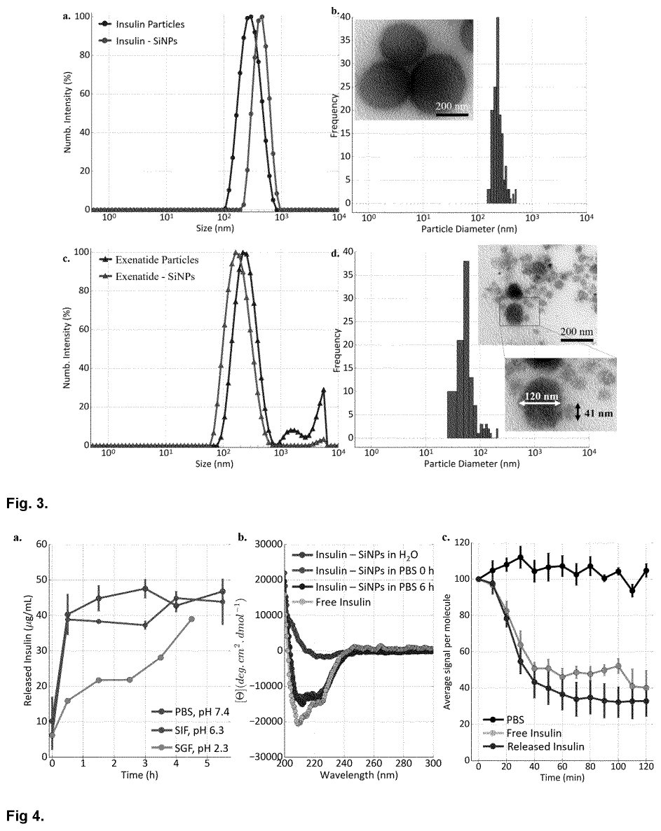

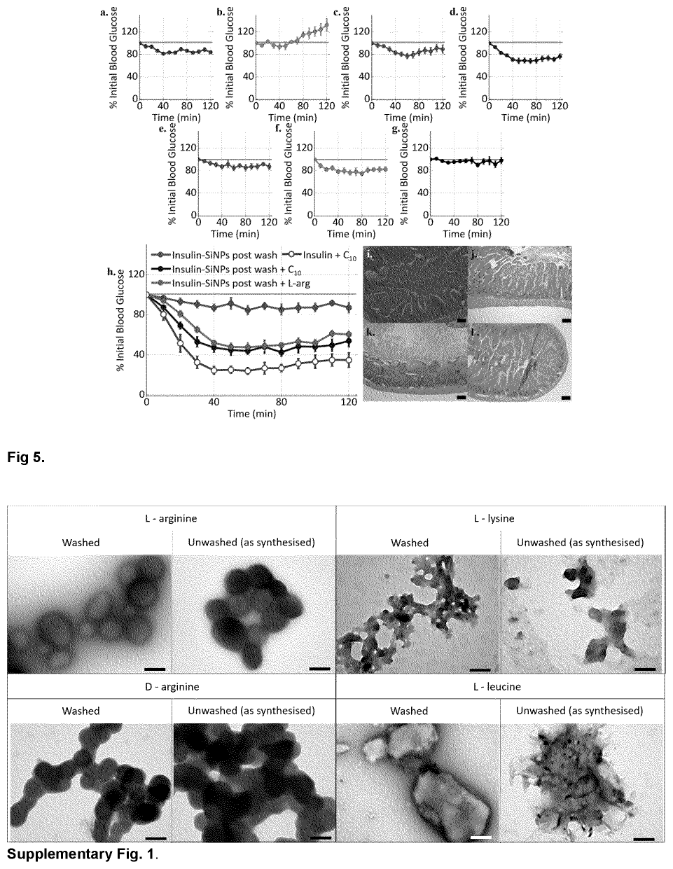

FIG. 1. is a schematic of SiNP synthesis for insulin. This pathway was also used for exenatide and BSA, but with no acid in Step 1 and using different reagent ratios.

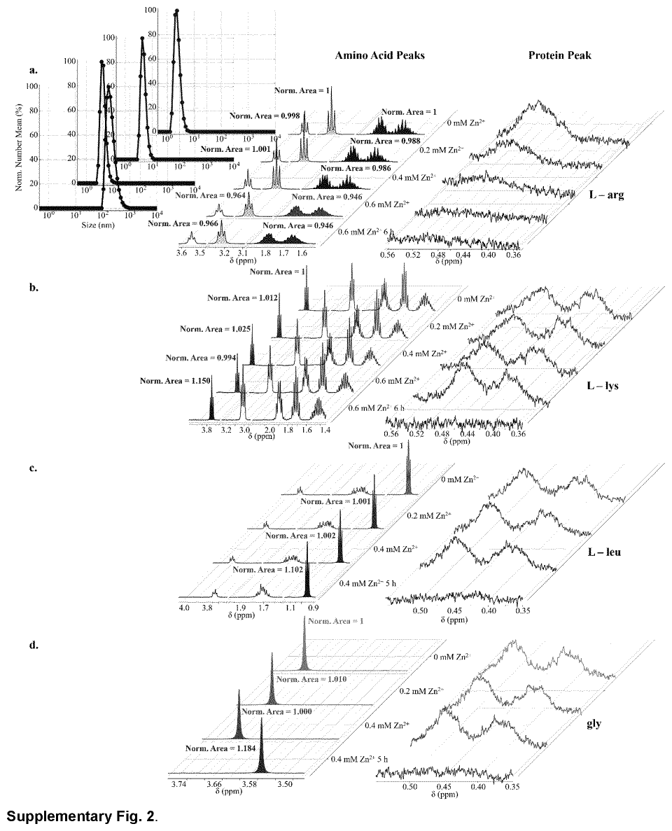

FIG. 2. Conditions affecting insulin core particle synthesis, as indicated by DLS. (a) pH of the insulin solution upon addition of ZnCl₂, (b) amino acid, (c) insulin concentration, (d) ratios of insulin: L-arg: ZnCl₂. The pH of the solution in (c) was adjusted to >6.5. The mean size and PdI values of experiments are given in Supplementary Table 1.

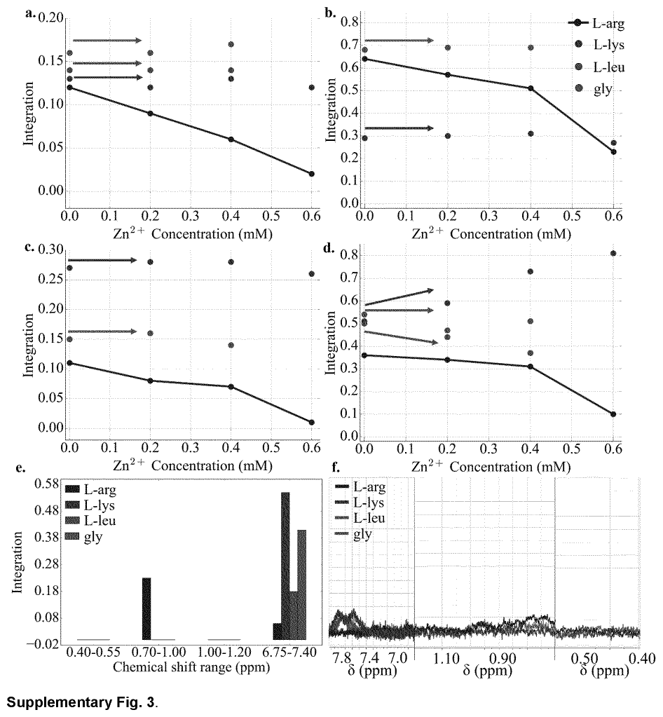

FIG. 3. Representative characterisation of an insulin-SiNP batch (a) size by DLS for core particles and coated particles (b) TEM. Insert in (b) shows particle morphology by TEM. (c) DLS and (d) TEM size and particle morphology for exenatide core particles and exenatide-SiNPs upon washing are shown.

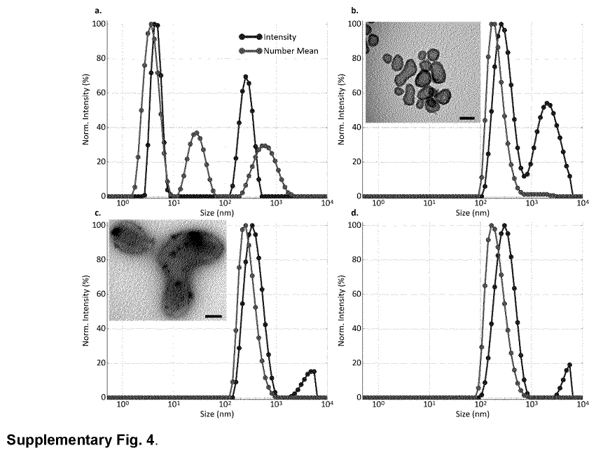

FIG. 4. (a) Dissolution of insulin-SiNPs in SGF, SIF, and PBS, N=3±SD (b) CD scan of the secondary structure of the SiNP-released insulin compared to free insulin. (c) Free insulin solution and SiNP-released insulin induced comparable blood glucose reductions in 6 rats injected by the s.c. route (1 IU/kg).

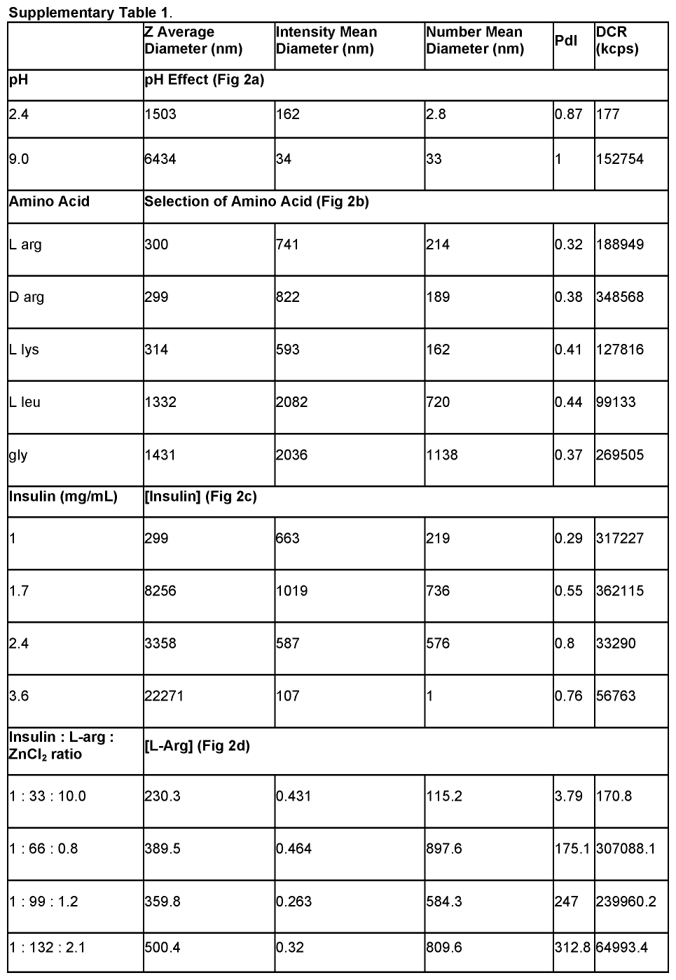

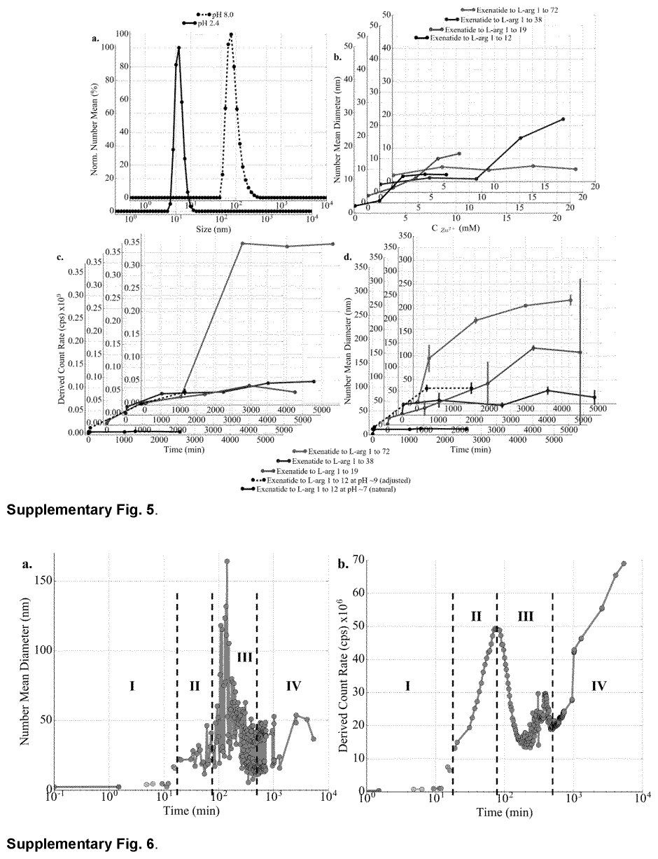

FIG. 5. Blood glucose levels of rats following intra-jejunal instillations of insulin-SiNPs. (a) insulin, (b) insulin+L-arg, (c) insulin core particles, (d) pre-washed insulin-SiNPs, (e) post-washed insulin-SiNPs, (f) supernatant, (g) PBS, (h) washed insulin-SiNPs, washed insulin-SiNPs+C₁₀ and washed insulin-SiNP+L-arg, insulin+C₁₀ ad-mixture. Histology of the jejunum after exposure to: (i) pre-washed insulin-SiNPs (j) washed insulin-SiNPs+L-arg, (k) washed insulin-SiNPs+C₁₀ and (l) insulin+C₁₀. Doses: insulin (50 I.U./kg), C₁₀ (100 mM), and L-arg (3 mg/kg). Values for each group given as Mean±SEM and n=4-10. The grey line in a-h indicates 100%. Scale bar in i-l is 100 μm.

Supplementary FIG. 1. TEM of insulin-SiNP made with four different amino acids in the core. Scale bar=200 nm.

Supplementary FIG. 2. Changes occurring in the insulin solution during particle synthesis when using (a) L-arg, (b) L-lys, (c) L-leu and (d) gly, as measured by ¹H NMR. The areas displayed are for the shaded peaks. Changes are shown for both the amino acid and a selected peak of the protein (0.3-0.4 ppm).

Supplementary FIG. 3. Shifts in integration of the insulin peaks at (a) 0.4-0.55, (b) 0.7-1.0, (c) 1.02-1.2 and (d) 6.75-7.42 with addition of ZnCl₂ (Zn²⁺), as observed by liquid ¹H NMR. The blue line is an eye guide. Coloured arrows in a-d show the general trend of the dataset for convenience of the reader. Also provided is the integration pattern of insulin after particle formation in terms of (e) integrated values and (f) spectra.

Supplementary FIG. 4. Distribution of insulin core particles made with increasing insulin: L-arg molar ratios and subsequent to addition of 0.1-0.3 mM ZnCl₂. (a) 1:33, (b) 1:66, (c) 1:99, (d) 1:132, as shown in FIG. 2d. 1:66 is the threshold ratio for particle formation. TEM is from a sample batch after TEOS addition, but not from the batches in the Figure. Scale=100 nm.

Supplementary Table 1. Size, PdI, and derived count rate (DCR) of insulin core particles (See. FIG. 2.)

Supplementary FIG. 5. Summary of factors impacting insulin core particle synthesis (a) pH of exenatide solution upon addition of ZnCl₂, (b) the insulin: L-arg:ZnCl₂ ratio. Change in particles distribution in terms of (c) the derived count rate (in thousands of cps) and (d) number mean diameter (nm). The attenuator of the DLS in (c and d) was pre-set to 8.

Supplementary FIG. 6. DLS monitoring by (a) number mean and (b) DCR of core particle formation kinetics of exenatide:L-arg:Zn=1:38:14 particles over 24 h. Stage I: Zn²⁺ was added at a rate of 10 μl/10 min until a critical concentration was reached and particles form. Stage II: Particle self-assembly starts as DCR and particle size increase. Stage III: Particle DCR and size decrease. Stage IV: No increase in particle size was observed, while there was an increase by a factor of >2 in DCR.

Supplementary FIG. 7. Change in the size of exenatide core particles at a dilution of 1 in 50 in water, expressed as change in the distribution by (a) intensity and (b) number. Changes over time in (c) Z average, (d) intensity and (e) number mean diameter, and (f) DCR are also presented. Particles rearrange themselves within 24 h when distribution becomes narrower and the final size is doubled, as observed by number mean.

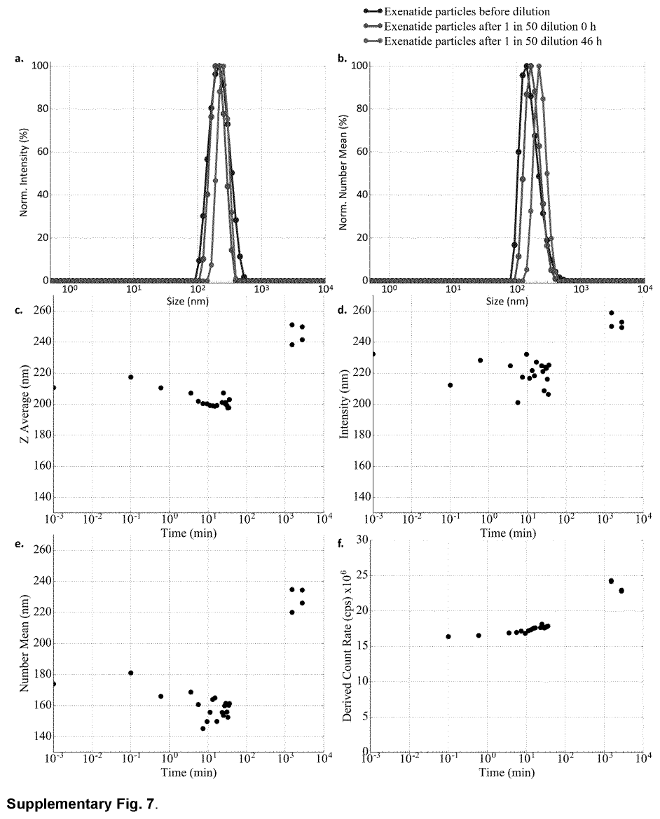

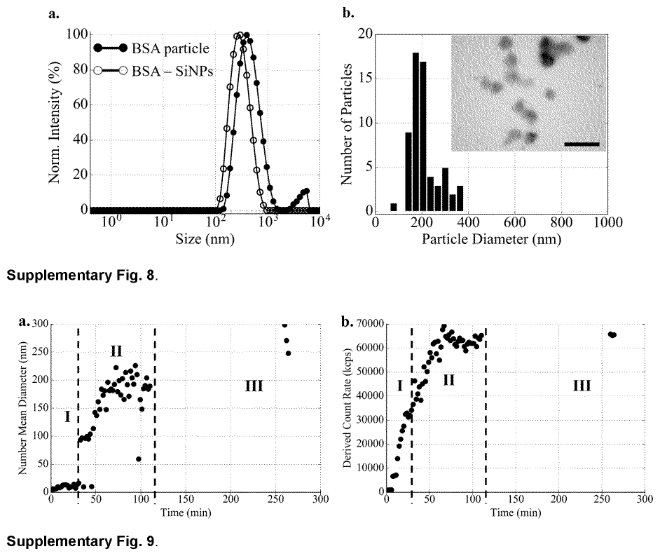

Supplementary FIG. 8. (a) Hydrodynamic diameter of BSA core particle and BSA-SiNPs in DLS and (b) an example TEM (insert) and histogram of BSA-SiNPs. Scale bar=500 nm.

Supplementary FIG. 9. Synthesis of BSA core particles over time expressed in (a) number mean diameter and (b) DCR. Stage I: Zn²⁺ concentration is increased, but no particle formation occurs. Stage II: Critical Zn²⁺ concentration is reached and particles start forming. After an initial increase in size, BSA particles start growing. Stage III: Particles continue to grow slowly for at least up to 24 h after synthesis initiation.

Supplementary FIG. 10. Representative TEMs of exenatide core particles and exenatide-SiNPs before and after washing. Scale bar=500 nm.

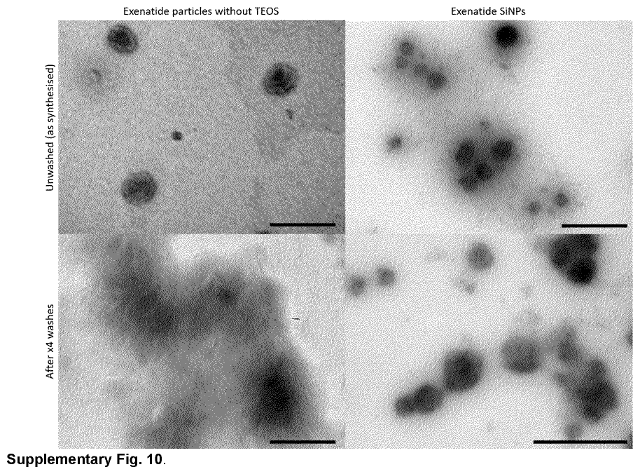

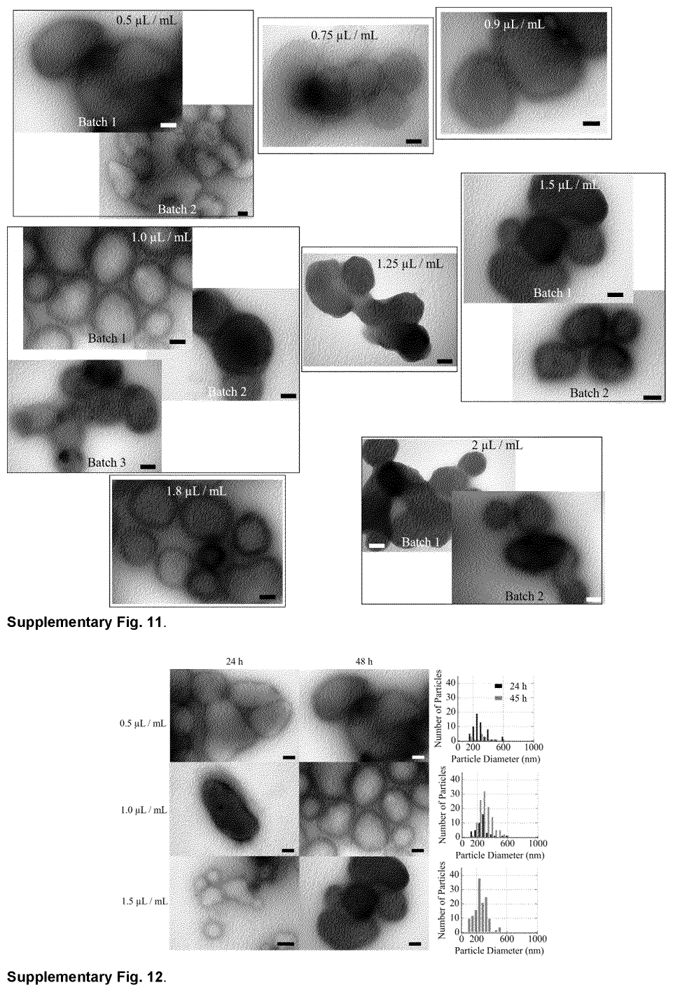

Supplementary FIG. 11. TEMs of insulin-SiNPs made with increasing TEOS volume. Cross-linking occurred at >40 h at pH˜9 with slow homogenization. An increase in shell diameter was seen when TEOS was added. After addition of 1.25 μL/mL, the core shell structure was often not observed. Several batches show reproducibility. Scale bars=100 nm.

Supplementary FIG. 12. Monitoring changes in morphology of the same particle batch from 24-48 h with different TEOS volumes. Scale bars=100 nm.

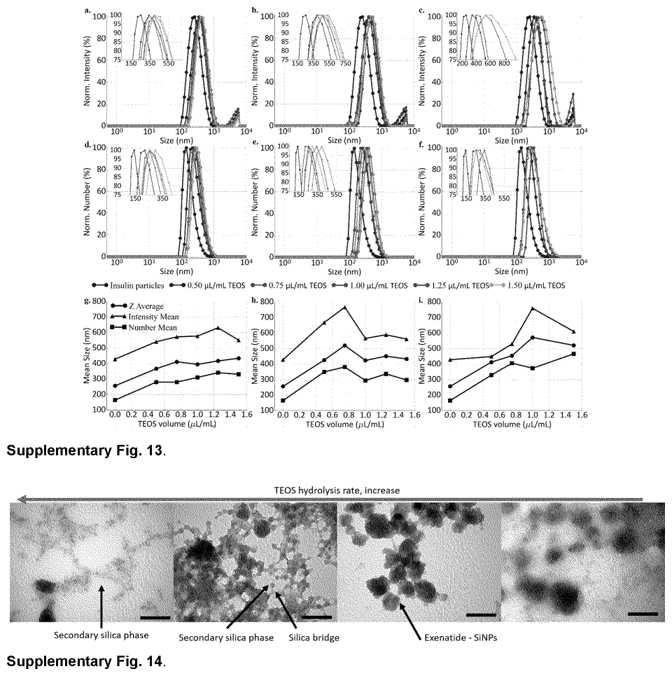

Supplementary FIG. 13. Size distributions of insulin-SiNPs with increasing TEOS volume added after (a and d) 17 h, (b and e) 38 h and (c and f) after washing with water. There is a weak dependence between TEOS volume and particle size as seen by (g) Z-average, (h) intensity mean and (i) number mean.

Supplementary FIG. 14. Change in the morphology of exenatide-SiNPs with an increase in silicic acid generation rate. At a high monomer generation rate, secondary silica particles appear. Scale bars=100 nm.

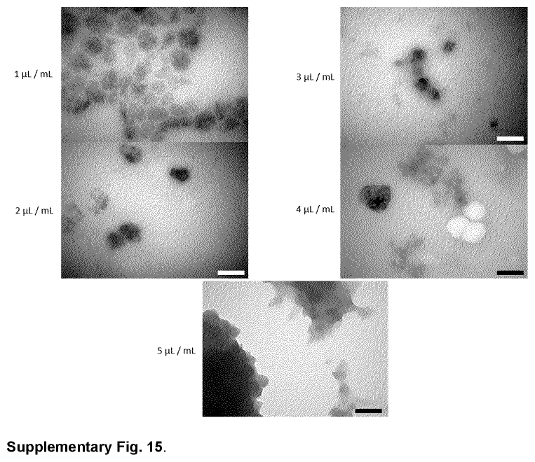

Supplementary FIG. 15. Change in morphology of exenatide-SiNPs with an increase in TEOS volume per mL of reaction. Scale bars=100 nm.

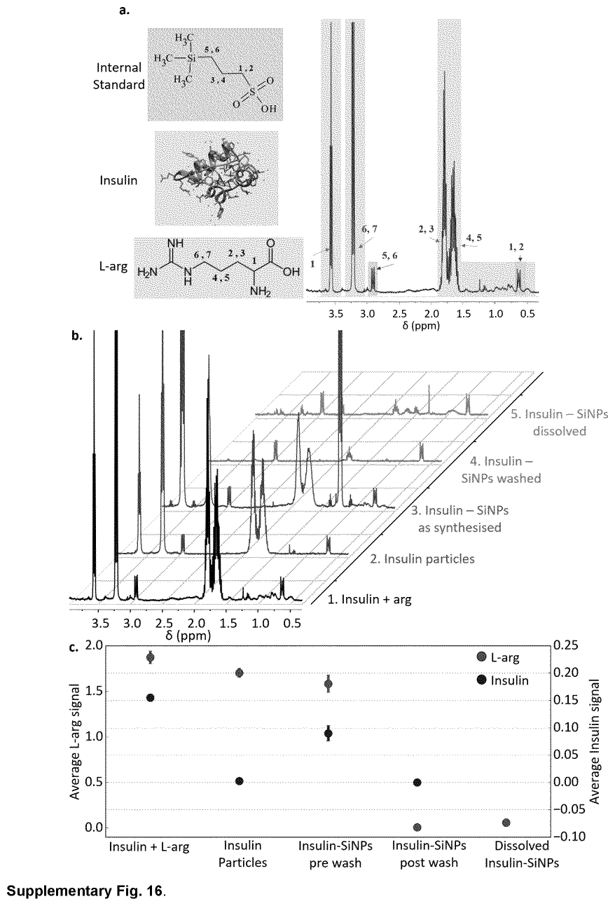

Supplementary FIG. 16. ¹H NMR monitoring of the signal changes during stages of particle synthesis. The signals are assigned in (a) using colour coding; (b) NMR signal at different stages of particle synthesis; (c) Changes in signal relate to L-arg (green) and insulin (blue).

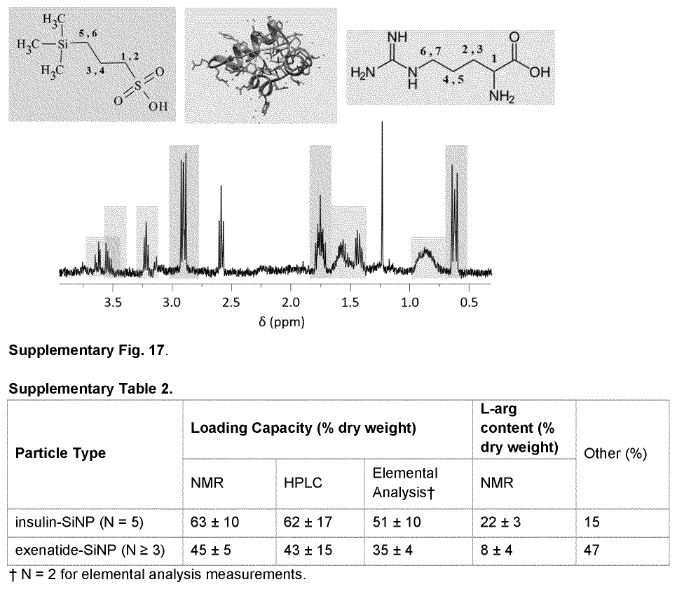

Supplementary FIG. 17. Assignment of the ¹H NMR signal after particle dissolution in 0.2 M NaOH at 37° C. for >2 h. Colour indicates molecular assignment.

Supplementary Table 2. Average loading capacity and L-arg content in insulin- and exenatide-SiNP.

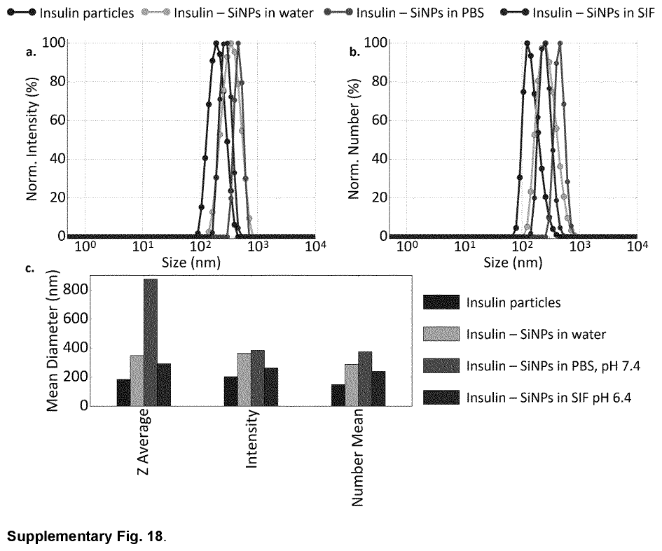

Supplementary FIG. 18. Size distribution of insulin-SiNPs in terms of (a) intensity and (b) number. (c) Further changes in the mean size are shown by Z-average, intensity and number mean diameter in water, PBS, and SIF compared with the initial insulin (core) particle batch.

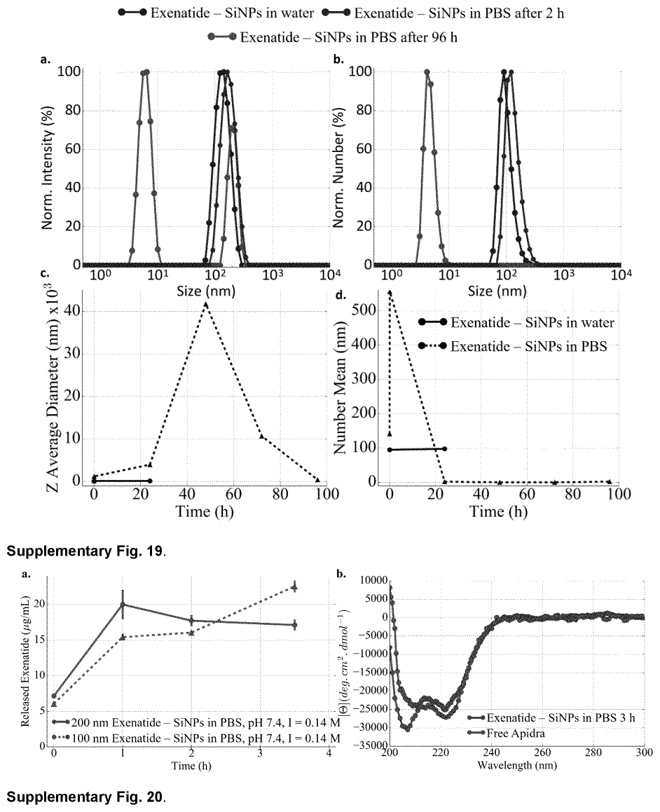

Supplementary FIG. 19. Size distribution of exenatide-SiNPs: (a) intensity, (b) number, in water, and PBS after 2 and 96 h. Further changes in the mean size are shown by (c) Z average and (d) number mean diameter. The figure shows relatively rapid aggregation of particles in PBS and over time dissolution to molecular state.

Supplementary FIG. 20. Exenatide-SiNPs (c) dissolution kinetics in PBS measured by HPLC and (b) the secondary structure of the released protein was measured with CD and compared to free exenatide.

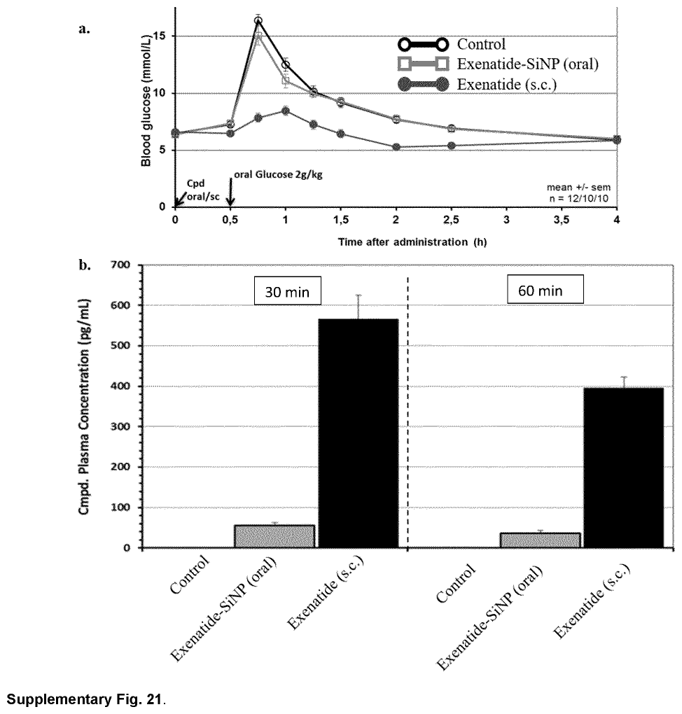

Supplementary FIG. 21. Oral gavage of exenatide-SiNP in mice and glucose challenge assay in response to exenatide-SiNP (1 mg/kg, p. o.) or exenatide (0.01 mg/kg, s.c). (a) a small PD-effect is seen in response to glucose challenge to gavaged exenatide-SiNP, but much less than that of free exenatide (s.c.). (b) Plasma concentrations at 30 and 60 min reflect a large PK difference between oral and s.c., nevertheless the gavaged SiNP delivered some exenatide.

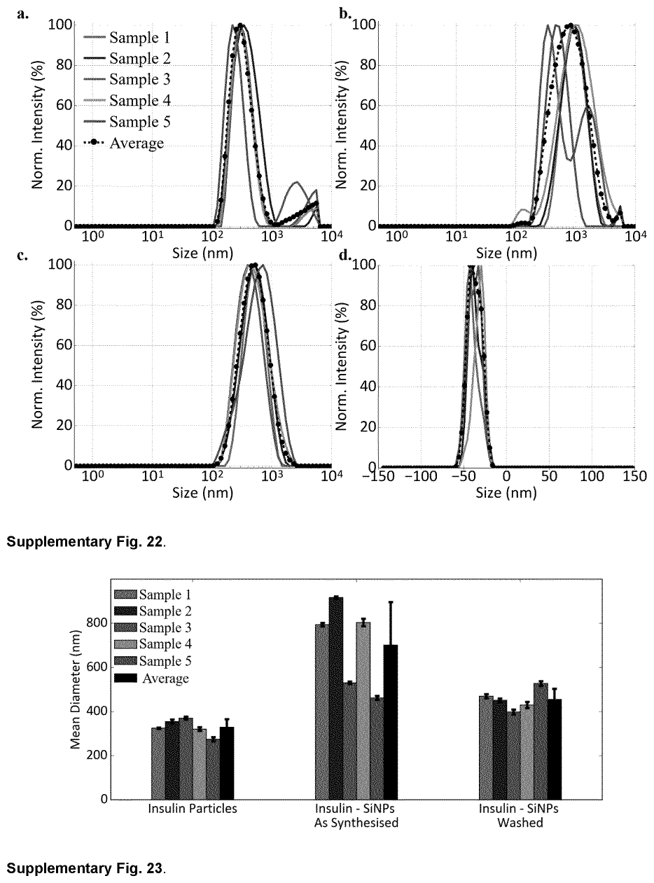

Supplementary FIG. 22. Size distributions of five scientific repeat batches (a) after insulin core particle synthesis, (b) after crosslinking and (c) after purification shown by DLS intensity. (d) Zeta potential of batches 1-4 in water 1 mM NaCl. Mean values of for all are shown in Supplementary Table S6. Particle content for the above samples is in Table 1 in main text.

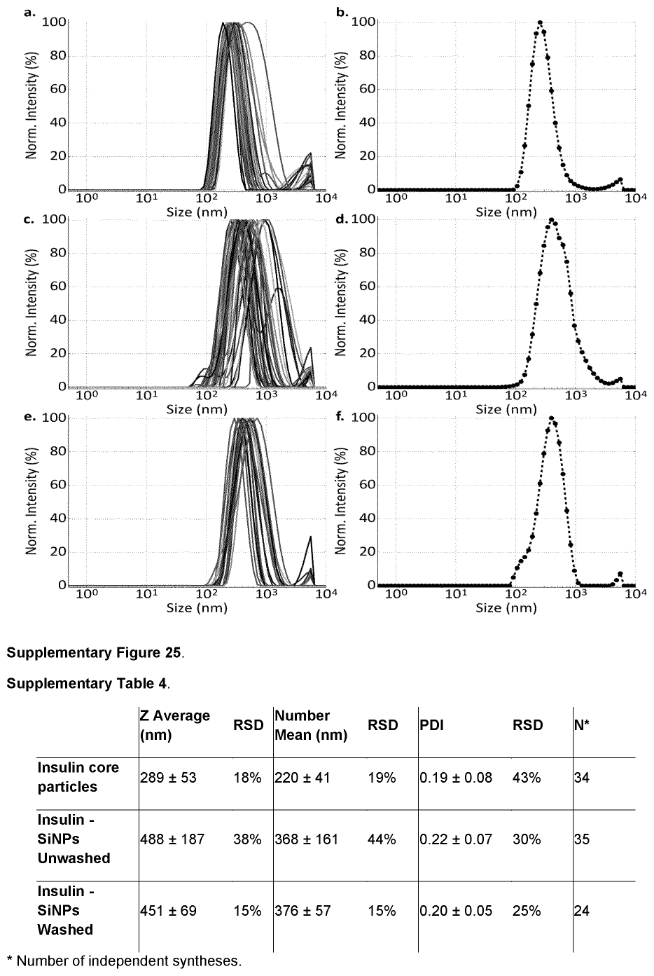

Supplementary FIG. 23. Mean hydrodynamic diameter (Z average) of five scientific repeats and their average. Samples are the same as in Supplementary FIG. 25 and Supplementary Table S6. Error bars represent SD.

Supplementary Table 3. Diameter and zeta potential mean values for particle reproducibility.

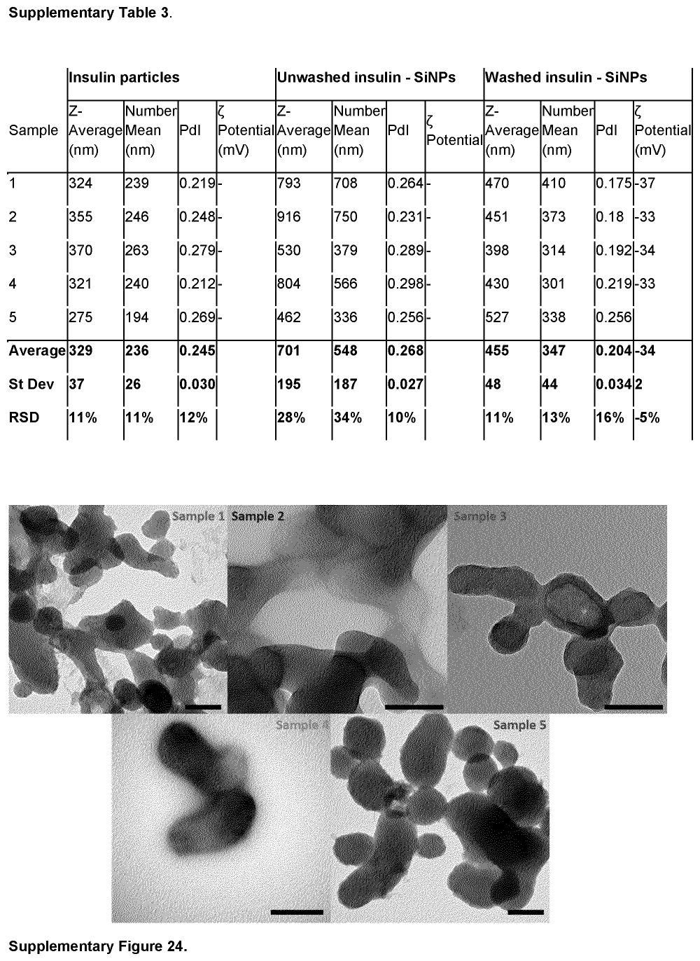

Supplementary FIG. 24. TEM images of scientific repeats 1-5 described in Supplementary FIG. 25, 26 and Supplementary Table S6. Scale bar=200 nm.

Supplementary FIG. 25. Overlapped size distributions and an averaged size distribution of most samples made using the same methodology for (a) and (b) insulin core particles, (c) and (d) unwashed insulin-SiNPs, and (e) and (f) washed insulin-SiNPs.

Supplementary Table 4. Average hydrodynamic diameters of insulin-SiNPs at key stages of synthesis.

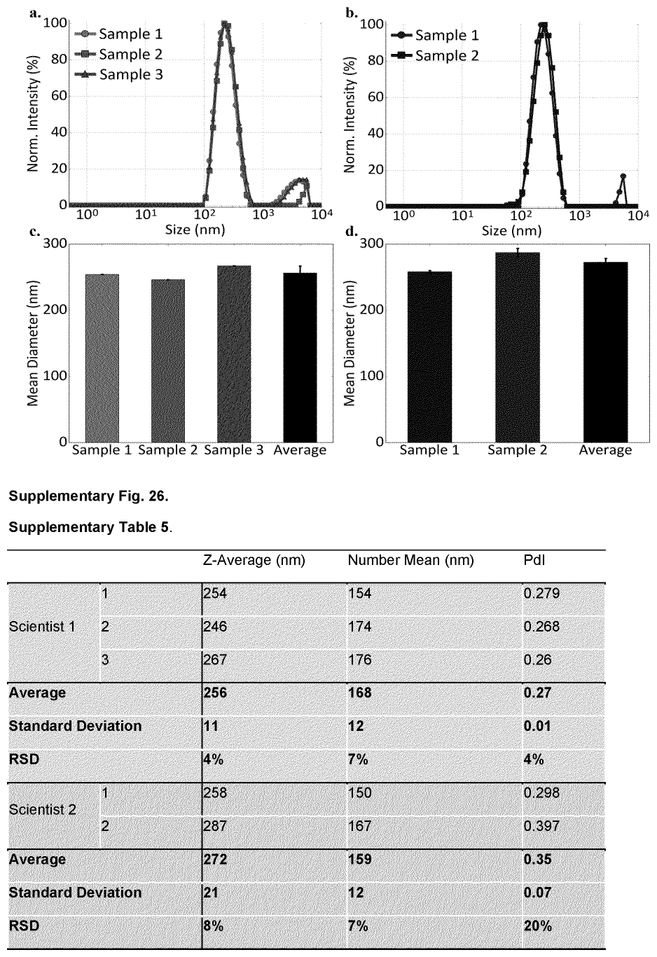

Supplementary FIG. 26. Reproducibility of insulin particle synthesis assessed by two operators. Size distribution of scientific repeats made by operators (a) 1 and (b) 2, and mean size of scientific repeats made by operators (c) 1 and (d) 2 are presented.

Supplementary Table 5. Hydrodynamic diameter as denoted by Z Average and Number Mean of samples made by two operators.

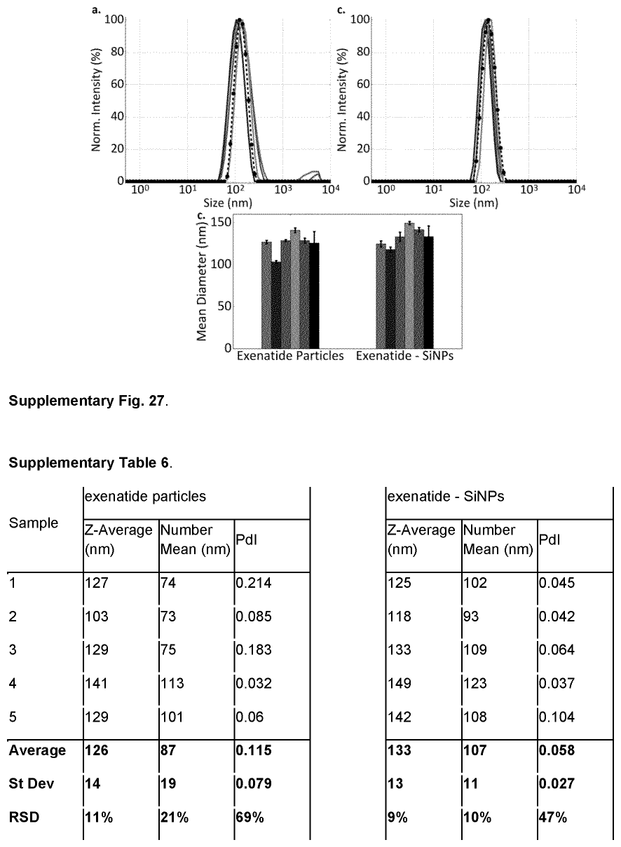

Supplementary FIG. 27. Overlapped size distributions of five independent batches of (a) exenatide core particles and (b) washed exenatide-SiNPs along with (c) mean diameter of the same particle batches.

Supplementary Table 6. Mean hydrodynamic diameter of five exenatide core particle and exenatide-SiNP batches.

Supplementary Table 7. Comparison of the content of five independent washed insulin-SiNP particle batches synthesised side-by-side.

Supplementary Table 8. Comparison of the mean sizes of the particle suspensions: data obtained at both UCD and Sanofi (Montpellier). One batch was divided and analysed.

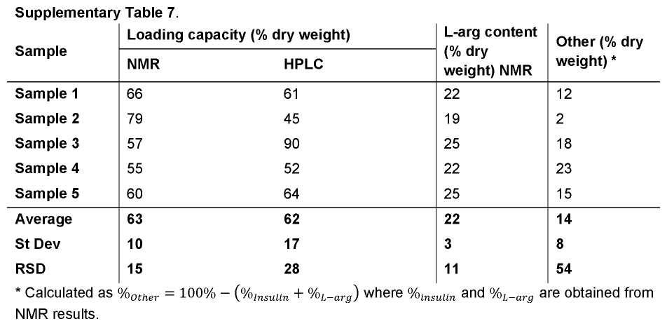

Supplementary FIG. 28. Particle size distributions measured by DLS at Sanofi (top) and UCD (bottom). The red solid line (highlighting the mode diameter) and the blue dashed lines (highlighting the edges of the distribution) help compare distributions.

Supplementary Table 9. Comparison of the mean zeta potentials of the exenatide-SiNP suspensions obtained at UCD and Sanofi.

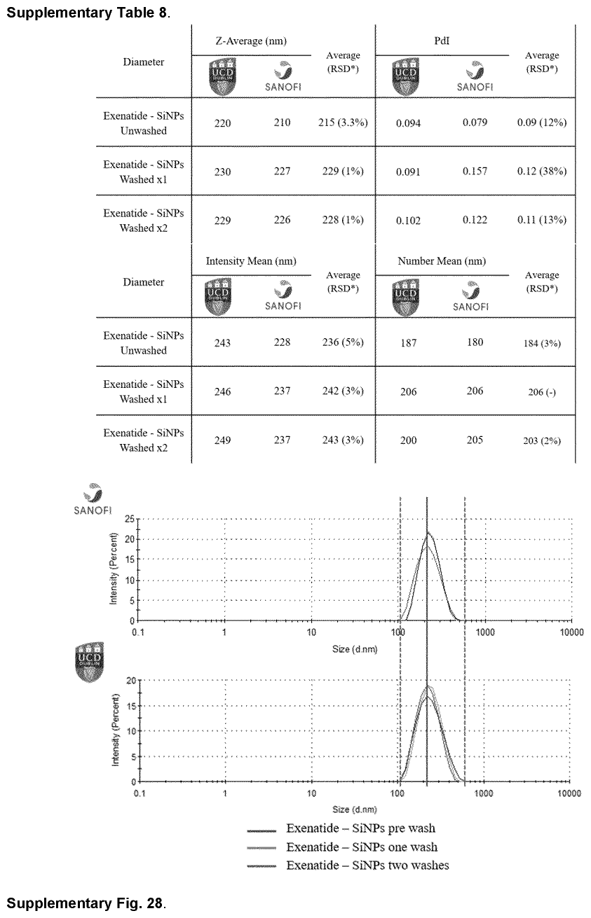

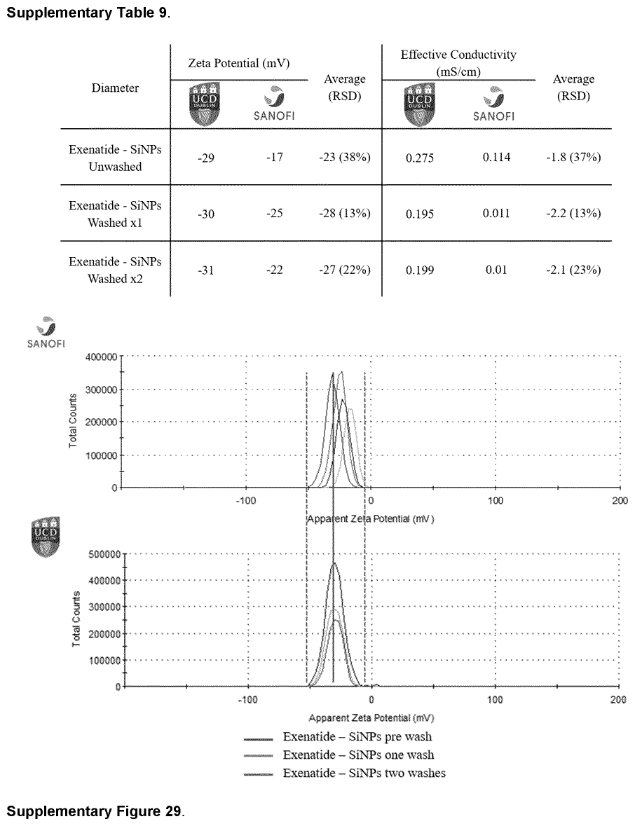

Supplementary FIG. 29. Particle size distributions measured by DLS at Sanofi (top) and UCD (bottom). The red solid line (highlighting the mode diameter) and the blue dashed lines (highlighting the edges of the distribution) help compare the distributions.

Supplementary Table 10. Comparison of the mean zeta potentials of the exenatide-SiNP suspensions obtained at UCD and Sanofi.

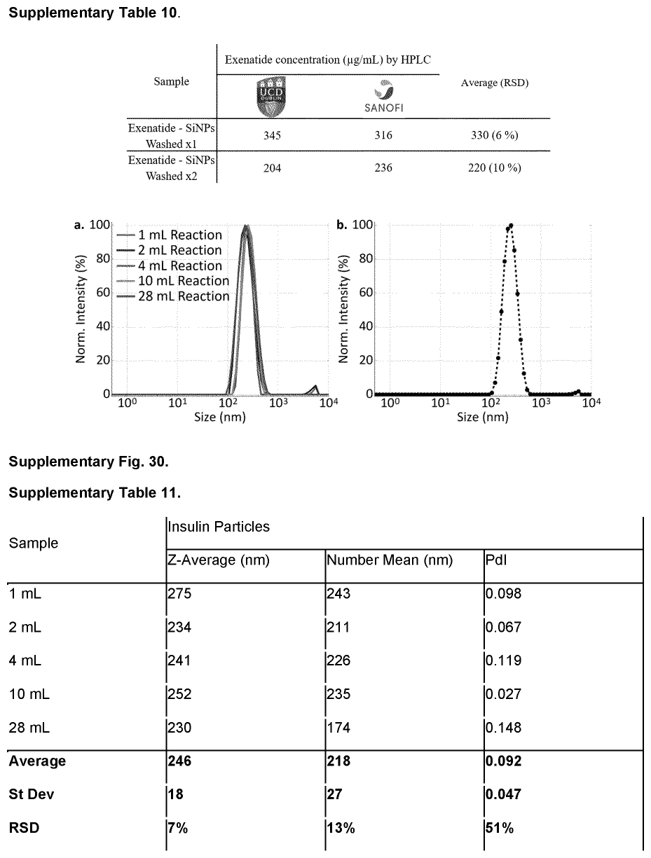

Supplementary FIG. 30. (a) Overlapped size distributions of insulin core particles made in increasing reaction volumes (1-28 mL) along with (b) a particle distribution averaged between them.

Supplementary Table 11. Mean hydrodynamic diameter of insulin core particles made in reactions of varying volume.

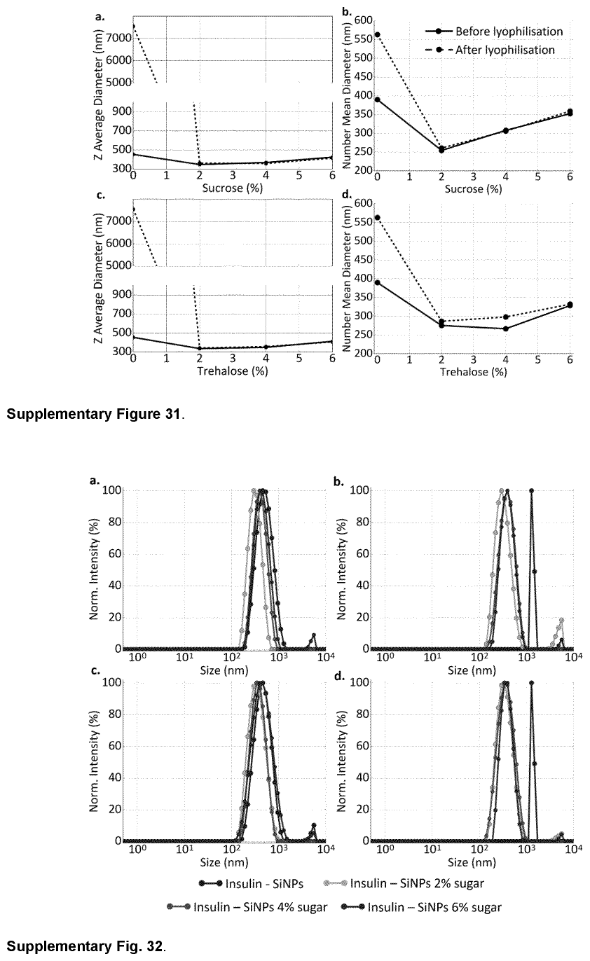

Supplementary FIG. 31. Change in the mean diameter of insulin-SiNPs stabilized with (a and b) sucrose or (c and d) trehalose, as measured by DLS. Both sugars facilitated particle preservation in lyophilised formats.

Supplementary FIG. 32. Change in the size of insulin-SiNPs stabilized with (a, b) sucrose or (c, d) trehalose, as measured by DLS.

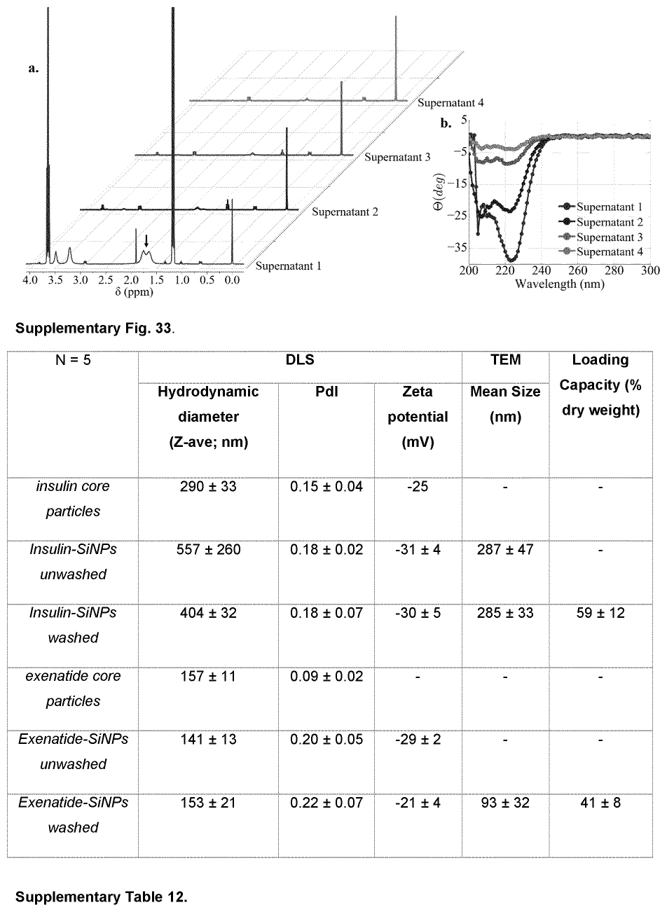

Supplementary FIG. 33. Supernatant analysis after washing insulin-SiNPs measured by (a) NMR and (b) CD. Arrow in a) indicates the L-arg peak. Samples are diluted 1 in 10 in CD. S1 in b) is too high a concentration for an accurate measurement.

Supplementary Table 12. Characterisation and loading of SiNP. 25° C. with fluorescence and HPLC, Concentration was 100 μg/ml under gentle stirring, SIF without pancreatin, Data shown as mean±SD (n=3).

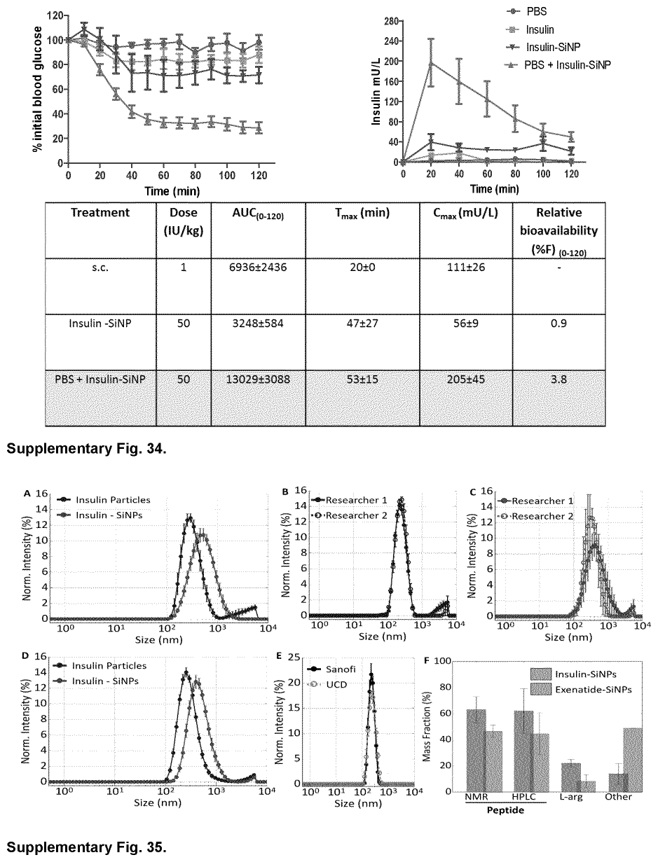

Supplementary FIG. 34. Insulin-SiNP: initial jejunal instillations reveal role of dispersion in PBS

Supplementary FIG. 35. Reproducibility of SiNP process. (A) Average size distributions of 5 side-by-side batches of insulin core particles and insulin-SiNPs. Average size distributions of (B) insulin core particles and (C) insulin-SiNPs made by two researchers (N=3 for researcher 1 and 2 for researcher 2). (D) Size distributions of insulin particles (N=34) and insulin-SiNPs (N=27) averaged from most synthesis done by one researcher in the project. (E) Comparison of the size distributions of the same exenatide particles and exenatide-SiNPs measured independently in two labs. (F) Content reproducibility of insulin- and exenatide-SiNPs. Error bars represent s.e. of the mean.

### RESULTS

Synthesis of SiNPs Comprised of a Protein Core Crosslinked with Silica

The aim was to develop a silica-coated core which would enable oral peptide delivery. Initially core particle formation was optimised using insulin glulisine (insulin, 5.1 kDa, Accession Number: DB01309), as a model peptide. Protein cores were also made using exenatide (4.8 kDa, Accession Number: P26349and bovine serum albumin (BSA, 69 kDa, Accession Number: P02769) as payloads. Synthesis comprised four stages, Step 1: peptide/protein dispersion, Step 2: L-arg addition, Step 3: ZnCl₂ addition, and Step 4: silica crosslinking (FIG. 1). The optimum reagent ratios and final physicochemical properties of the construct depended on payload selection. Step 1 largely depended on the hydrophobicity of the payload (FIG. 1). Insulin has low solubility at neutral pH and was therefore dissolved in HCl (10 mM), whereas exenatide and BSA were dissolved in water. L-arg was then added to the payload solution in molar ratios (Step 2, FIG. 1). For insulin, a final molar ratio of 1 (insulin):99 (L-arg) was optimal, while for exenatide and BSA it was ˜1:40. The molar ratio, volume, and rate of ZnCl₂ addition (Step 3) was dependent on payload. In Step 3, insulin core particle formation took <60 min, whereas exenatide and BSA particle formation took >12 h. In Step 4, the core particles were then cross-linked with silica to generate SiNPs. Precursor volumes of 1-2 μL tetraethyl orthosilicate (TEOS) per mL particle solution were optimal in forming the coating.

Synthesis of Insulin Core Nanoparticles

The pH, the type of amino acid, and insulin concentration affected formation. Particles did not form if the pH was <7, while large aggregates formed when pH>10 (FIG. 2a). Selected amino acids were investigated as excipients to prevent protein aggregation^{38, 39}. The effects of the basic amino acids: L- and D-arg and L-lysine (lys), the neutral amino acid:glycine (gly), and the hydrophobic amino acid:L-leucine (leu) on insulin particle synthesis were compared. The molar ratio between the insulin and the selected amino acids was set at 1:99 because stable particles had been produced using this ratio with L-arg. Rapid aggregation of insulin was observed when neutral and hydrophobic amino acids were used, resulting in formation of micron-sized particles. 15% of insulin particles made with gly and L-leu, and 10% made with L-lys were larger than 1 μm, whereas <99% of those made with L- and D-arg were in the micron range (FIG. 2b). The morphology of the final silica shell cross-linked insulin core particles (insulin-SiNPs) was also dependent on amino acid selection: particles formed with either L- and D-arg had a regular spherical shape, while those formed using the other three amino acids were asymmetrical as revealed by TEM (Supplementary FIG. 1). These results were confirmed by ¹H NMR: L-arg had a complex interaction with insulin, while the other amino acids had comparably simpler profiles suggesting weaker interaction (Supplementary FIGS. 2 and 3, and Supplementary files). Moreover, when using L-lys, L-leu, and gly, the pH had to be adjusted to >8 with NaOH (0.1 M, 5-20 μL) in Step 3, which was not ideal for peptide stability⁴⁰. The amine-rich side chain in L-arg is likely to play a key role in insulin particle formation and stabilization. In summary, L- and D-arg and, to some extent L-lys, favoured formation of a stable sub-micron core particle population. The natural L-form of arg was selected for all subsequent studies.

An insulin concentration of >1 mg/mL led to visible uncontrolled particle aggregation confirmed by DLS (FIG. 2c). Here, the insulin:L-arg ratio was also 1:99 and the pH was ˜9. There was a complex relationship between the insulin:L-arg molar ratio with respect to the ZnCl₂ critical concentration at which particles start forming. An increase in the insulin:L-arg molar ratio required a matching increase in the ZnCl₂ critical concentration (FIG. 2d), and this led to an increase in size of the core particles as measured by DLS and TEM (Supplementary FIG. 4). Though both 1:66 and 1:99 ratios of insulin:L-arg can be used to synthesise core particles, the latter ratio led to more reproducible particles with mean diameters of 290±33 nm. Factors including the reaction volume impacted upon particle synthesis and changes were made to compensate. For volume reactions of >2 mL, homogenization was required during and between additions of the two ZnCl₂ aliquots. For volume reactions >10 mL, a stirring rate of >600 rpm was used during Step 3 during 4-6 additions of ZnCl₂, followed by a stir rate of <400 rpm during particle formation. Changing the concentration of HCl in Step 1 also affected the critical ZnCl₂ concentration. Observations on other factors impact on the synthesis can be found in the notes section of the Supplementary file. Mean sizes and PdIs for all experiments in FIG. 2 are presented in Supplementary Table 1.

Synthesis of Exenatide Core Particles

Though some of the factors impacting insulin core particle formation also applied to exenatide, there were differences. In contrast to insulin, exenatide is soluble in water and did not need pre-dispersion in HCl and could be used at a higher initial concentration of 3 mg/mL. Similar to insulin, a pH range of 7-10 was required for particle synthesis (Supplementary FIG. 5a) and the physicochemical properties of exenatide particles were also dependent on the L-arg and ZnCl₂ concentrations (Supplementary FIG. 5b). Exenatide core particles with mean diameters of 10-200 nm were synthesised using an exenatide:L-arg molar ratio range from 1:19 (i.e. 78.9 μg/mL:1.5 mg/mL) to 1:72 (i.e. 12.5 μg/mL:9.0 mg/mL), and with an exenatide:ZnCl₂ ratio of 1:9 (Supplementary FIG. 5). However, core exenatide particle formation took 10-30 h compared to ˜1 h for insulin (Supplementary FIGS. 5c, d and 6). Assembly of exenatide core particles was more complex than for insulin, (Supplementary FIG. 6). After exenatide dispersion in water (step 1) and L-arg addition (step 2) ZnCl₂ was added to the exenatide/L-arg solution at a rate of 10 μL/10 min until particles formed. Particle growth was observed to be a complex process and could be described in four stages (Supplementary FIG. 6). Stage I comprised the slow addition of ZnCl₂ to the protein solution until the critical concentration was reached. The size of the core particles in the exenatide solution increased after each addition as observed by DLS, and then stabilized within 10 min. Stage II started when the critical ZnCl₂ concentration was reached. First, an increase in size was initially observed at an exenatide:ZnCl₂ratio of 1:7, followed by a steady fivefold increase in signal (Supplementary FIG. 6b). Although the dispersion signals were not of sufficient quality to precisely determine particle size, the majority of the dispersion was <100 nm in diameter with a PdI<0.4 (Supplementary FIG. 6a). In stage III the signal reduced and the PdI increased, indicating aggregation. After 8 h the PdI of the dispersion was reduced and a single peak of ˜100 nm diameter was observable (stage IV). In total, the time of formation of exenatide core particles was ˜16 hours. In addition, there was also a difference in the optimal critical molar ratio of the peptide to ZnCl₂:˜1:4 for insulin and ˜1:10 for exenatide. This suggests a possible difference in the driving force as well as composition and cohesion of the final protein core particle. Indeed, when diluted, the dispersion of exenatide particles changed over time (Supplementary FIG. 7). Several factors likely contributed to the differences in the synthesis of insulin and exenatide core particles: (i) protein physicochemical properties, e.g. size and/or hydrophobicity; (ii) difference in initial reagent concentrations; (iii) subtle differences in peptide structure, including the relative abundance and protonation state of specific functional groups.

Synthesis of BSA Core Particles and Initial Summary

BSA was also used as a model for protein-based particles⁴¹. We synthesised BSA core particles with a mean diameter of 430 nm using conditions and kinetics similar to those used for exenatide core particles (Supplementary FIGS. 8 and 9). The morphology of the final BSA-SiNPs were comparable to that of the final exenatide-SiNPs (insert, Supplementary FIG. 8). The BSA results suggest that protein solubility in water is a major factor in core formation. BSA exhibited relatively slow assembly (>5 h), and it required the highest critical ZnCl₂ molar ratio of the three payloads (1:153, BSA:ZnCl₂). Insulin, exenatide, and BSA core particles of controlled reproducible size were synthesised. Managing pH and the molar ratios of payload:L-arg: ZnCl₂ were crucial to particle formation and size control. The strength of payload interactions with L-arg seems to be a major factor in synthesis. Insulin required the lowest critical ZnCl₂ molar ratio (1:4, insulin:ZnCl₂), and particle formation was rapid.

Crosslinking Core Particles with Silica to Make SiNPs

For oral administration, core particles need to be stabilized to protect payload structure and functionality and to allow controlled release. Silica is known to condense around particulates⁴² and therefore we hypothesised that silica crosslinking might be suitable for coating and stabilizing core particles entrapping payload.

Properties of Insulin- and Exenatide-Entrapped SiNPs

Properties were studied after core particle formation, after crosslinking (SiNPs, pre-centrifugation wash step) and after purification (SiNPs, i.e. washed twice by centrifugation and resuspension). Silica coating of core particles was confirmed by TEM, whereas uncoated particles fell apart during TEM preparation and had a diameter twice of that measured by DLS (Supplementary FIG. 10). The morphology of the final SiNP product was dependent upon the initial TEOS volume/mL, its speed of hydrolysis, and the payload in the core particles. Increasing the TEOS volume/mL or the hydrolysis rate dissipated the core-shell morphology of insulin-SiNPs and caused formation of a secondary silica phase in the exenatide-SiNPs and a reduction of the loading capacity of the final crosslinked particle (Supplementary FIG. 11-15 and Supplementary files). Incubation of 1 μl/mL TEOS for 48 h was optimal for insulin-SiNPs synthesis, while 2 μg/mL over 24 h was optimal for exenatide-SiNPs, both processed carried out at 25° C. and pH˜9. The hydrodynamic diameter of the insulin-SiNPs changed with crosslinking from ˜290 nm in the core particle to ˜400 nm as measured by DLS (˜280 nm by TEM) (FIG. 3a, b; Table 1). Exenatide dispersions were unchanged in diameter with TEOS addition (FIG. 3c, Table 1), possibly due to a broadening sample size distribution which was bimodal in some cases and which displayed a different morphology (FIG. 3d, Table 1). This suggests that the composition of insulin- and exenatide-SiNPs is different, possibly due to hydrophobic interactions in the initial insulin particles. NMR was also used to monitor core particle synthesis and crosslinking. While ˜100% of the insulin and ˜9% of the L-arg were associated with insulin particles, about 50% of insulin was quickly released upon particle crosslinking (Supplementary FIG. 16c). This could be due to a more complex structure of core particles with an inner core where interactions are strong, and an outer layer with more loosely bound insulin. Full observations are in Supplementary FIGS. 16, 17 and associated files.

TABLE 1
Characterisation of insulin- and exenatide-SiNPs.
Loading capacity and standard
deviation are an average of all samples and all methods used.

| | DLS | | | | |
|---|---|---|---|---|---|
| | Hydro- | | | TEM | Loading |
| | dynamic | | Zeta | Mean | Capacity |
| | diameter | | potential | Size | (% dry |
| N = 5 | (Z-ave; nm) | PdI | (mV) | (nm) | weight) |
| insulin core | 290 ± 33 | 0.15 ± | −25 | — | — |
| particles | | 0.04 | | | |
| Insulin- | 557 ± 260 | 0.18 ± | −31 ± 4 | 287 ± | — |
| SiNPs | | 0.02 | | 47 | |
| unwashed | | | | | |
| Insulin- | 404 ± 32 | 0.18 ± | −30 ± 5 | 285 ± | 59 ± 12 |
| SiNPs | | 0.07 | | 33 | |
| washed | | | | | |
| exenatide | 157 ± 11 | 0.09 ± | — | — | — |
| core | | 0.02 | | | |
| particles | | | | | |
| Exenatide- | 141 ± 13 | 0.20 ± | −29 ± 2 | — | — |
| SiNPs | | 0.05 | | | |
| unwashed | | | | | |
| Exenatide- | 153 ± 21 | 0.22 ± | −21 ± 4 | 93 ± | 41 ± 8 |
| SiNPs | | 0.07 | | 32 | |
| washed | | | | | |

Loading Capacity of Insulin- and Exenatide-SiNPs.

HPLC and NMR were used to determine SiNP loading capacity, while elemental analysis was used in some studies for confirmation. Insulin-SiNPs and exenatide-SiNPs contained >40% of protein by weight depending on synthesis conditions (Supplementary Table 2). The former had a loading capacity of 59%, and the latter had a loading capacity of 41% (Table 1). Insulin-SiNPs and exenatide-SiNPs both had loading efficiency of ˜50%, as determined by NMR, HPLC, Micro Bicinchoninic Acid (BCA) protein assay, circular dichroism (CD), and protein fluorescence labelling (see Supplementary files for methods).

Particle Behaviour in Complex Conditions

The behaviour of insulin-SiNPs and exenatide-SiNPs in biologically-relevant buffers depended on media pH and salinity. Both constructs aggregated in PBS, while remaining colloidally-stable in simulated small intestinal fluid (SIF) at pH 6.3 (Supplementary FIGS. 18, 19 for insulin-SiNPs and exenatide-SiNPs, respectively). PBS was used to measure the concentration of the released peptide, while SIF was used to examine secondary structure. For insulin-SiNPs (˜300 nm in diameter, particle concentration of 0.2 mg/mL), release was measured by HPLC in simulated gastric fluid (SGF) at pH 2.3, SIF at pH 6.3, and PBS at pH 7.4. Particle dissolution was more rapid with an increase in pH; samples dissolved within 30 min when pH>6, while they did not reach a release plateau over 4.5 h at pH 2.3 (FIG. 4a). Released insulin had a secondary structure comparable to that of the free insulin in the same conditions, as measured by CD (FIG. 4b). Exenatide-SiNP dissolved in PBS in a size-dependent fashion: particles of ˜90 nm in diameter dissolved within 60 min at a particle concentration 0.2 mg/mL, while ˜200 nm diameter particles took over 3 h (Supplementary FIG. 20a). This difference was expected, as the rate of dissolution was dependent upon sample surface area and the mass-normalized 90 nm particles have a larger surface area than 200 nm particles in PBS. Similarly, the released exenatide had a secondary structure comparable to that of the free peptide (Supplementary FIG. 20b).

Bioactivity of Released Insulin from SiNPs Following Sub-Cutaneous Administration to Rats

Bioactivity of SiNP-released insulin was confirmed by sub-cutaneous (s.c.) injection of the released material from washed SiNP to normal rats. A reduction in blood glucose was observed after injection of SiNP-released insulin at a dose of 1 IU/kg, which was comparable to that seen upon injection of free insulin solution at the same dose (FIG. 4c, d). This proves that SiNP-released insulin is bioactive in vivo.

Efficacy of Insulin-SiNPs Following Rat Jejunal Instillations

Insulin-SiNPs were instilled into rat jejunal loops and resulting blood glucose changes were measured in response to a dose of 50 IU/kg. Insulin-SiNPs were assessed at most stages of synthesis, including insulin-arginine solution, as well as unwashed and washed insulin-SiNPs. This also included the supernatant from the insulin-SiNP wash to account for any changes due to the protein aggregation step (FIG. 5a-g). While the pre-centrifuged unwashed insulin-SiNPs achieved ˜75% blood glucose reduction, none of the other iterations were efficacious. We hypothesised that loosely-associated L-arg and insulin were attached to the particle surface before washing and that their interaction led to permeation enhancement of the subsequently-released insulin from the unwashed SiNP. Since the washed insulin-SiNPs released bioactive insulin, as determined by the blood glucose reduction in response to s.c. administration (FIG. 4c), these instilled particles likely also released it in the jejunum, but it was unable to cross the epithelium without permeability enhancement.

To investigate this further, we mixed washed insulin-SiNPs with both a known permeation enhancer for oral peptides, sodium caprate (C₁₀)⁴³ and separately, L-arg. Both resulted in a reduction of blood glucose comparable to that of s.c.-injected insulin and free insulin mixed with C₁₀ (FIG. 5h). These data prove that insulin was released from the washed insulin-SiNPs, but it could not cross the jejunal epithelium without assistance of L-arg. In the unwashed scenario, there is enough L-arg and insulin weakly associated with the particle to enable this. Histological examination showed mild cell sloughing of tissues exposed to Insulin-SiNPs, but it was less than that seen in tissue exposed to SiNPs with the additional enhancers (FIG. 5i-l).

The exenatide-SiNPs were also assessed an oral glucose challenge mouse model following oral delivery at a 1 mg/kg dose. Exenatide (10 μg/kg) abolished the challenge-induced blood glucose excursion following s.c. administration, whereas the effect of the orally-administered unwashed- and washed exenatide-SiNPs was modest (Supplementary FIG. 21). Pharmacokinetic data from mouse plasma confirmed pharmacodynamic effects of both oral and s.c. administration. Exenatide (s.c.) prevented the change from baseline in both AUC and Cₘₐₓ by 77% relative to control, whereas the values were 16% and 15% for the oral exenatide-SiNP (Supplementary FIG. 21). Exenatide-SiNPs were therefore bioactive in a rodent model by the oral route, albeit far less than free exenatide (s.c.). It is important to note that intestinal presentation of the exenatide-SiNPs to the mice was not optimised.

Discussion

The current study demonstrated the synthesis, characterisation, and initial proof-of-concept in rodent model for an oral peptide nanoparticle construct based on a core of payload and the excipients, L-arg and ZnCl₂, stabilized and coated with a silica shell. Silica presents a matrix which maintains particle structure in the presence of acidic pH, and consequently, intestinal enzymes, but which then dissolves as the pH rises in the small intestine³⁴. Stable crosslinked particles were formed, with properties which depended on the payload. Additionally, we examined over 20 factors in the formation of the core particles and final SiNP constructs. Notable features of the SiNP include high peptide loading capacity, an advantage over many nanoparticle prototypes, even when they have a high association efficiency, e.g.⁴⁴. Without this, too much payload will be required for a practical oral formulation. The insulin in SiNP was released at a pH value present in the small intestine and was structurally intact. Both insulin- and exenatide-SiNPs were biologically functional.

We have studied the synthesis, physicochemical and in vivo reproducibility of our constructs in an attempt to adhere to recent recommendations on nanoparticle characterisation⁴⁵. The reproducibility of the synthesis was thoroughly investigated across many batches (Supplementary files). Synthesis with insulin has been carried out by five researchers, some with minimal training. To briefly summarize, the variability in measuring the size expressed as relative SD of a single insulin core particle batch in DLS was ˜3%. Comparison of 5 batches made in the same study yielded a size variability for the insulin core particle of ˜11%, the pre-wash insulin-SiNPs of ˜28% and the washed insulin-SiNPs of ˜11% (Supplementary FIGS. 22-24 and Supplementary Table 3). The higher size variability of pre-washed SiNP was attributed to the possible co-existence of several insulin forms in the sample. Somewhat higher variability was noted when comparing all samples (>20) synthesised using the same procedure by a single researcher: 18% for insulin core particles, 38% and 15% for insulin-SiNPs pre-washed and washed, respectively (Supplementary FIG. 25 and Supplementary Table 4). Finally, a comparison between the syntheses of two independent researchers, producing a total of 5 samples, yielded an overall 6% size variability in the final insulin-SiNP (Supplementary FIG. 26 and Supplementary Table 5). A variability of ˜20% was measured for loading capacity of the particles (Supplementary Table 7). A similar result was obtained for exenatide core and -SiNPs (Supplementary FIG. 27 and Supplementary Table 6). More details are available in ESI. Characterisation reproducibility was confirmed between two independent labs where variability in particle size and loading was within expected margins (Supplementary FIGS. 28 and 29 and Supplementary Tables 8 to 10).

Bioactivity of both the insulin released from insulin-SiNPs and that of the insulin-entrapped in SiNPs was preserved. The former was done by comparing released- and free insulin injected by the s.c. route to rats, while the latter was achieved by assessing the blood glucose reduction of both unwashed insulin-SiNP and washed insulin-SiNPs admixed with either L-arg or C₁₀ in the rat jejunal instillation model. Furthermore, the in vivo results for instilled insulin-SiNP were reproducible across multiple batches. Finally, in anticipation of translation to large animal studies, synthesis scalability and storage through lyophilisation and reconstitution were established (Supplementary FIGS. 30-32 and Supplementary Table 11).

The final washed insulin-SiNPs required permeation enhancement in order to reduce plasma glucose to levels comparable with that induced by the s.c.-injected insulin, albeit at 50-fold higher doses. This suggests that, in addition to established mechanisms for L-arg of stabilising insulin through promotion of monomer formation⁴⁶, as well as prevention of protein aggregation in liquid formulations⁴⁷, an additional role of L-arg identified here is to act as a biocompatible permeability enhancer in the SiNP. While L-arg was recently discovered to be an intestinal permeation enhancer when ad-mixed with an insulin solution and administered to rat ileal loops in vivo³⁷, to our knowledge this is the first example of its capacity to perform an equivalent role as part of an insulin nanoparticle construct. It is possible to supplement L-arg during lyophilisation, which may both further stabilize the SiNPs and augment permeation enhancement for an optimised formulation.

It is often overlooked that most other nanoparticle constructs also include excipients, but their precise roles have not been clarified. Indeed, in one of the first prototypes used for oral insulin in the 1980s, the bile salt, sodium deoxycholate, and poloxamer 188 were included in polyalkycyanoacrylate nanoparticles and these were effective in diabetic rats⁴⁸. More recently, a nanocapsule construct also intended for oral insulin comprised the known permeation enhancers, protamine, and sodium glycocholate²⁰, yielding plasma glucose reductions upon intestinal instillation to rats. A third, albeit less efficacious, example was a polyethylene glycol (PEG)-polyglutamic acid polymeric nanocapsule entrapping insulin attached to a cell penetrating peptide, octa-arginine, in turn conjugated to cholesterol or the medium chain fatty acid enhancer, lauric acid¹⁹. Finally, the excipient and known enhancer, Tween®-80,⁴⁹ was included in the oily core of a polyarginine-coated nanocapsule designed to deliver salmon calcitonin across Caco-2 monolayers, primarily taking advantage of poly-arginine's capacity to act as a cell penetrating peptide⁵⁰. In each of these examples, the excipients preserved peptide stability against pancreatic enzymes, but demonstration of permeation enhancement by them was never proven, nor a mechanism suggested.

Evidence that the majority of these and similar nanoparticles are internalised to a great extent by intestinal epithelia either in vitro or in vivo is equivocal^{51, 52}; many prototypes seem to get stuck in intestinal mucus, while those with hydrophilic coatings may permeate mucus to reach the epithelial wall⁵³. A reasonable conclusion therefore is, once the particle negotiates the mucus, there is a permeation enhancement effect from the L-arg excipient when co-released with the peptide in high concentration close to the epithelium. In the current study, unwashed insulin-SiNP were effective in rat jejunal instillations, while the washed particles were largely ineffective. This would suggest that in the unwashed state, insulin and L-arg are both concentrated in the particle core and are weakly associated with the silica shell, with some also free in solution. This was confirmed by NMR. It is the latter two sources that are removed from the construct upon washing by centrifugation, as suggested by the NMR and CD results (Supplementary FIG. 33). This is also consistent with a 20% reduction in plasma glucose following instillation of the supernatant from the centrifugation step, which contained released free insulin and L-arg. Finally, when L-arg was added back to the washed insulin-SiNP, the glucose-lowering effect was once again conferred, though higher than that seen with the unwashed SiNP. The conclusion therefore is that L-arg acted as an enhancer for the remaining insulin in the particles after the washing step, by itself this released insulin was not sufficient to generate a pharmacodynamic response since it cannot cross the epithelium without assistance. That this concept was more general was evident from almost identical in vivo data achieved with the gold standard PE, C₁₀, both when ad-mixed with free insulin and with washed insulin-SiNPs.

Conclusions

Insulin-, exenatide- and BSA-SiNPs were synthesised using a simple scalable process with potential for oral delivery. We elucidated the fundamental principles of core particle formation and the physicochemical properties of the final entrapped SiNP products, as well as ensuring synthesis and characterisation reproducibility. Prototype batches were made reproducibly and were characterised in accordance with recent recommendations. Attractive features were the high payload loading as well as high proportion of insulin release at small intestinal pH values. The insulin and exenatide-SiNPs were bioactive following intestinal instillation and oral gavage to rats and mice respectively. The mechanism of action for the insulin-SiNP relies not on epithelial particle uptake, but on the co-entrapped excipient, L-arg, acting as a PE to enable the particle-released payload to cross the epithelium. Though the work relates to three payloads, we expect it to be applicable to other peptides and proteins. It is also expected that nanoparticle constructs with a high loading capacity may be useful for applications beyond oral peptide and protein delivery, including local delivery to arthritic joints⁵⁴. The use of silica as a stabilizing matrix in multiple formats may also be further enhanced by functionalisation for targeted delivery to diseased tissue receptors⁵⁵.

### REFERENCES

1. de la Torre, B. G., Albericio, F. The pharmaceutical industry in 2017. An analysis of FDA drug approvals from the perspective of molecules. Molecules 23, pii: E533 (2018).
2. Moroz, E., Matoori, S., Leroux, J. C. Oral delivery of macromolecular drugs: where we are after almost 100 years of attempts. Adv. Drug Deliv. Rev. 101,108-121 (2016).
3. McLenon, J., Rogers, M. A. M. The fear of needles: a systematic review and meta-analysis. J. Adv. Nurs. August 14. doi: 10.1111/jan.13818 (2018).
4. Strasburger, C. J., Karavitaki, N., Störmann, S., Trainer, P. J., Kreitschmann-Andermahr, I., Droste, M., Korbonits, M., Feldmann, B., Zopf, K., Sanderson, V. F., Schwicker, D., Gelbaum, D., Haviv, A., Bidlingmaier, M., Biermasz, N. R. Patient-reported outcomes of parenteral somatostatin analogue injections in 195 patients with acromegaly. Eur. J. Endocrinol. 174, 355-362 (2016).
5. Owens, D. R., Monnier, L., Barnett, A. H. Future challenges and therapeutic opportunities in type 2 diabetes: Changing the paradigm of current therapy. Diabetes Obes. Metab. 19, 1339-1352 (2017).
6. Gedawy, A., Martinez, J., Al-Salami, H., Dass, C. R. Oral insulin delivery: existing barriers and current counter-strategies. J. Pharm. Pharmacol. 70, 197-213 (2018).
7. Shao, J., Zaro, J. L., Shen, W. C. Tissue barriers and novel approaches to achieve hepatoselectivity of subcutaneously-injected insulin therapeutics. Tissue Barriers 4, e1156804. (2016).
8. Halberg, I. B., Lyby, K., Wassermann, K., Heise, T., Zijlstra, E., Plum-Mörschel, L. Efficacy and safety of oral basal insulin versus subcutaneous insulin glargine in type 2 diabetes: a randomised, double-blind, phase 2 trial. Lancet Diabetes Endocrinol. 7, 179-188 (2019).
9. Rosenstock, J., Allison, D., Birkenfeld, A. L., Blicher, T. M., Deenadayalan, S., Jacobsen, J. B., et al. Effect of additional oral semaglutide vs sitagliptin on glycated hemoglobin in adults with Type 2 diabetes uncontrolled with metformin alone or with sulfonylurea: The PIONEER 3 randomized clinical trial. JAMA. doi: 10.1001/jama.2019.2942. (2019).
10. Nielsen, D. S., Shepherd, N. E., Xu, W., Lucke, A. J., Stoermer, M. J., Fairlie, D. P. Orally-absorbed cyclic peptides. Chem. Rev. 117, 8094-8128 (2017).
11.Aguirre, T. A. Teijeiro-Osorio, D., Rosa, M., Coulter, I. S., Alonso, M. J., Brayden D. J. Current status of selected oral peptide technologies in advanced preclinical development and in clinical trials. Adv. Drug Deliv. Rev. 106, 223-241 (2016).
12. Anselmo, A.C., Gokarn, Y., Mitragotri, S. Non-invasive delivery strategies for biologics. Nat. Rev. Drug Discov. doi: 10.1038/nrd.2018.183 (2018).
13.Buckley, S. T., Bækdal, T. A., Vegge, A., Maarbjerg, S. J., Pyke, C., Ahnfelt-Rønne, J., Madsen, K. G., Schéele, S. G., Alanentalo, T., Kirk, R. K., Pedersen, B. L., Skyggebjerg, R. B., Benie, A. J., Strauss, H. M., Wahlund, P. O., Bjerregaard, S., Farkas, E., Fekete, C., Søndergaard, F. L., Borregaard, J., Hartoft-Nielsen, M. L., Knudsen, L. B. Transcellular stomach absorption of a derivatized glucagon-like peptide-1 receptor agonist. Sci. Transl. Med. 10, pii: eaar7047. doi: 10.1126/scitranslmed.aar7047. (2018)
14. Melmed, S., Popovic, V., Bidlingmaier, M., Mercado, M, van der Lely, A. J., Biermasz, N., Bolanowski, M., Coculescu, M., Schopohl, J., Racz, K., Glaser, B., Goth, M., Greenman, Y., Trainer, P., Mezosi, E., Shimon, I., Giustina, A., Korbonits, M., Bronstein, M. D., Kleinberg, D., Teichman, S., Gliko-Kabir, I., Mamluk, R., Haviv, A., Strasburger, C. Safety and efficacy of oral octreotide in acromegaly: results of a multicenter phase III trial. J. Clin. Endocrinol. Metab. 100, 1699-1708 (2015).
15. McCartney, F., Gleeson, J. P., Brayden, D. J. Safety concerns over the use of intestinal permeation enhancers: a mini-review. Tissue Barriers. 4:e1176822 (2016).
16. Maisel, K., Ensign, L., Reddy, M., Cone, R., Hanes, J. Effect of surface chemistry on nanoparticle interaction with gastrointestinal mucus and distribution in the gastrointestinal tract following oral and rectal administration in the mouse. J. Control. Release 197:48-57 (2015).
17. Griesser, J., Hetényi, G., Kadas, H., Demarne, F., Jannin, V., Bernkop-Schnürch, A. Self-emulsifying peptide drug delivery systems: how to make them highly mucus permeating. Int. J. Pharm. 538,159-166 (2018).
18. Niu, Z., Tedesco, E., Benetti, F., Mabondzo, A., Montagner, I. M, Marigo, I., et al. Rational design of polyarginine nanocapsules intended to help peptides overcoming intestinal barriers. J. Control. Release. 263:4-17 (2017),
19. Niu, Z., Samaridou, E., Jaumain, E., Coëne, J., Ullio, G., Shrestha, N., Garcia, J., Durán-Lobato, M., Tovar, S., Santander-Ortega, M. J., Lozano, M. V., Arroyo-Jimenez, M. M., Ramos-Membrive, R., Peñuelas, I., Mabondzo, A., Préat, V, Teixidó, M., Giralt, E., Alonso, M. J. PEG-PGA enveloped octaarginine-peptide nanocomplexes: An oral peptide delivery strategy. J. Control. Release. 276,125-139 (2018).
20. Thwala, L. N., Delgado, D. P., Leone, K., Marigo, I., Benetti, F., Chenlo, M., Alvarez, C. V., Tovar, S., Dieguez, C., Csaba, N. S., Alonso, M. J. Protamine nanocapsules as carriers for oral peptide delivery. J. Control. Release 291, 157-168 (2018).
21. Santalices, I., Torres, D., Lozano, M. V., Arroyo-Jiménez, M. M., Alonso, M. J., Santander-Ortega, M. J. Influence of the surface properties of nanocapsules on their interaction with intestinal barriers. Eur J Pharm Biopharm. 133:203-213 (2018).
22. Dapergolas, G., Gregoriadis, G. Lancet. Hypoglycaemic effect of liposome-entrapped insulin administered intragastrically into rats. Lancet, 2(7990):824-827 (1976).
23. Damgé, C., Michel, C., Aprahamian, M., Couvreur, P. New approach for oral administration of insulin with polyalkylcyanoacrylate nanocapsules as drug carrier. Diabetes 37, 246-251 (1988).
24.Pridgen, E. M., Alexis, F., Kuo, T. T., Levy-Nissenbaum, E., Karnik, R., Blumberg, R. S., Langer, R., Farokhzad, O. C. Transepithelial transport of Fc-targeted nanoparticles by the neonatal fc receptor for oral delivery. Sci. Transl. Med. 5, 213ra167 (2013).
25. Kaklotar, D., Agrawal, P., Abdulla, A., Singh, R. P., Mehata, A. K., Singh, S., Mishra, B., Pandey, B, L, Trigunayat, A., Muthu, M. S., Transition from passive to active targeting of oral insulin nanomedicines: enhancement in bioavailability and glycemic control in diabetes. Nanomedicine (Lond). 11(11):1465-1486 (2016).
26. Bitar, A., Ahmad, N. M., Fessi, H., Elaissari, A. Silica-based nanoparticles for biomedical applications. Drug Discov. Today. 17,1147-1154 (2012).
27. Vol, A., Gribova, O., Shamir, O., Levy, E., Ben Ishai, P., Gutina, A. G., Feldman, Y. Dielectric properties of a novel colloidal oral matrix drug carrier. Colloids Surf. B Biointerfaces. 155, 223-228 (2017).
28. Andreani, T., Miziara, L., Lorenzón, E. N., de Souza, A. L., Kiill, C. P., Fangueiro, J. F., Garcia, M. L., Gremião, P. D., Silva, A. M., Souto, E. B., Effect of mucoadhesive polymers on the in vitro performance of insulin-loaded silica nanoparticles: Interactions with mucin and biomembrane models. Eur. J. Pharm. Biopharm. 93,118-126 (2012).
29. Andreani, T., de Souza, A. L., Kiill, C. P., Lorenzón, E. N., Fangueiro, J. F., Calpena, A. C., Chaud, M. V., Garcia, M. L., Gremião, M. P., Silva, A. M., Souto, E. B. Preparation and characterization of PEG-coated silica nanoparticles for oral insulin delivery. Int. J. Pharm. 473, 627-635 (2014).
30. Zhao, X., Shan, C., Zu, Y., Zhang, Y., Wang, W., Wang, K., Sui, X., Li, R. Preparation, characterization, and evaluation in vivo of Ins-SiO₂-HP55 (insulin-loaded silica coating HP55) oral delivery of insulin. Int. J. Pharm. 454, 278-284 (2013).
31. Xu, C., Lei, C., Yu, C. Mesoporous silica nanoparticles for protein protection and delivery. Front. Chem. 7, 290 (2019). 10.3389/fchem.2019.00290.
32. Homayun, B., Lin, X., Choi, H. J. Challenges and recent progress in oral drug delivery systems for biopharmaceuticals. Pharmaceutics;11(3). pii: E129. doi: 10.3390/pharmaceutics11030129.(2019).
33.Tan, X., Liu, X., Zhang, Y., Zhang, H., Lin, X., Pu, C., Gou, J., He, H., Yin, T., Zhang, Y., Tang, X. Silica nanoparticles on the oral delivery of insulin. Expert Opin. Drug Deliv. 15, 805-820 (2018).
34.Giovaninni, G., Moore, C. J., Hall, A. J., Byrne, H. J., Gubala, V. pH-Dependent silica nanoparticle dissolution and cargo release. Colloids Surf. B Biointerfaces 169, 242-248. (2018).
35. Soudry-Kochavi L, Naraykin N, Di Paola R, Gugliandolo E, Peritore A, Cuzzocrea S, Ziv E, Nassar T, Benita S. Pharmacodynamical effects of orally administered exenatide nanoparticles embedded in gastro-resistant microparticles. Eur. J. Pharm. Biopharm. 133, 214-223 (2018).
36. Březina, K., Duboué-Dijon, E., Palivec, V., Jiráček, J., Křížek, T., Viola, C. M. et al., Can arginine inhibit insulin aggregation? A combined protein crystallography, capillary electrophoresis, and molecular simulation study. J. Phys. Chem. B. 122:10069-10076 (2018).
37. Kamei, N., Khafagy, E. S., Hirose, J., Takeda-Morishita, M. Potential of single cationic amino acid molecule “Arginine” for stimulating oral absorption of insulin. Int. J. Pharm. 521,176-183.
38. Ohtake, S., Kita, Y., Arakawa, T. Interactions of formulation excipients with proteins in solution and in the dried state. Adv. Drug Deliv. Rev. 63, 1053-73 (2011).
39. Stärtzel, P. Arginine as an excipient for protein freeze-drying: a mini review. J. Pharm. Sci. 107, 960-967 (2018).
40. Zhang, Y., Casabianca, L. B. Probing amino acid interaction with a polystyrene nanoparticle surface using saturation-transfer difference (STD)-NMR. J. Phys. Chem. Lett. 9: 6921-6925 (2018).
41. Kallar, A. R., Muthu, J., Selvam, S. Bioreducible amino acid-derived polymeric nanoparticles for delivery of functional proteins. Colloids Surf. B Biointerfaces 164, 396-405 (2018).
42. Tarn, D., Ashley, C. E., Xue, M., Carnes, E. C., Zink, J. I., Brinker, C. J. Mesoporous silica nanoparticle nanocarriers: biofunctionality and biocompatibility. Acc. Chem. Res. 46, 792-801 (2013).
43. Maher, S., Leonard, T. W., Jacobsen, J., Brayden, D. J. Safety and efficacy of sodium caprate in promoting oral drug absorption: from in vitro to the clinic. Adv. Drug Deliv. Rev. 61, 427-449 (2016).
44. Thwala, L. N., Beloqui, A., Csaba, N. S., et al. The interaction of protamine nanocapsules with the intestinal epithelium: A mechanistic approach. J. Control. Release. 243, 109-120 (2016).
45. Faria, M., Björnmalm, M., Thurecht, K. J. et al. Minimum information reporting in bio-nano experimental literature. Nature Nanotechnology, 13,777-785 (2018).
46. Kildegaard, J., Buckley, S. T., Nielsen, R. H., Povlsen, G. K., Seested, T., Ribel, U., Olsen, H. B., Ludvigsen, S., Jeppesen, C. B., Refsgaard, H. H. F., Bendtsen, K. M., Kristensen, N. R., Hostrup, S., Sturis, J., Elucidating the mechanism of absorption of fast-acting insulin aspart: the role of niacinamide. Pharm. Res. 36, 49 (2019).
47. Shiraki, K., Kudou, M., Fujiwara, S., et al, Biophysical effect of amino acids on the prevention of protein aggregation. J. Biochem. 132, 591-595 (2002).
48. Damge, C., Michel, C., Aprahamian, M., Couvreur, P. New approach for oral administration of insulin with polyalkylcyanoacrylate nanocapsules as drug carrier. Diabetes. 37, 246-251 (1988).
49. Maher, S., Mrsny, R. J., Brayden, D. J. Intestinal permeation enhancers for oral peptide delivery. Adv Drug Deliv Rev. 106, 277-319 (2016).
50. Gonzalez-Paredes, A., Tones, D., Alonso, M. J. Polyarginine nanocapsules: A versatile nanocarrier with potential in transmucosal drug delivery. Int. J. Pharm. 529, 474-485 (2017).
51. Date, A. A., Hanes, J., Ensign, L. M. Nanoparticles for oral delivery: Design, evaluation and state-of-the-art. J. Control. Release 240, 504-526 (2016).
52. Lundquist, P., Artursson, P. Oral absorption of peptides and nanoparticles across the human intestine: Opportunities, limitations and studies in human tissues. Adv. Drug Deliv. Rev. 106, 256-276 (2016).
53. Maisel, K., Ensign, L., Reddy, M, et al. Effect of surface chemistry on nanoparticle interaction with gastrointestinal mucus and distribution in the gastrointestinal tract following oral and rectal administration in the mouse. J. Control. Release 197, 48-57 (2015).
54. Brown, S., Kumar, S., Sharma, B. Intra-articular targeting of nanomaterials for the treatment of osteoarthritis. Acta Biomater. 2019 Mar. 9. pii: S1742-7061(19)30176-X. doi: 10.1016/j.actbio.2019.03.010.
55. Fortuni B, Inose T, Ricci M, et al. Polymeric engineering of nanoparticles for highly efficient multifunctional drug delivery systems. Sci. Rep. 9, 2666 (2019).

Supplementary Notes

Synthesis of Core Particles

¹H NMR to Assess Amino Acid-Insulin Interactions

Liquid ¹H NMR was also used to assess the role of the amino acid in insulin particle formation. Changes in the signal line-shape and integration with variation of the amino acid and its integration relative to insulin at several steps during ZnCl₂ addition were analysed. Such shifts in the NMR signal chemical shift (mode value) or full width half-maximum (FWHM) are attributed to changes in the molecule environment or its mobility¹.

Relating to Changes in Insulin Signal:
(i) Most of the insulin signal was lost when particles formed (as measured by DLS), indicating that most of the insulin is associated with the particle aggregate (Supplementary FIG. 2).
(ii) Line-shape of the insulin peak with L-arg had only one peak between 0.35 and 0.7 ppm, while it had two peaks when incubated with the other amino acids (Supplementary FIG. 2a). Similarly, other peaks were shifted or broadened with different amino acids.
(iii) Changes in the integration of insulin peaks for L-arg reduce with addition of ZnCl₂. However, they remain mostly constant prior to particle formation when other amino acids were used (Supplementary FIG. 3a-d).
(iv) There were amino acid-based changes in the insulin peak with ZnCl₂ addition (Supplementary FIG. 3a-d), including after particle formation (Supplementary FIGS. 3e and f). In the latter case, insulin incubated with the selected amino acids, (except L-arg), had a peak between 7.3 and 8.0 ppm.

Relating to Changes in Amino Acid Signal:
(i) Particle formation caused the integration of L-arg peaks to decrease by ˜5% and loss of fine structure, suggesting that a proportion of the L-arg is engaged in interacting with the insulin particles and insulin-SiNPs (Supplementary FIG. 2a).
(ii) Integration of other amino acids increased with ZnCl₂ addition (Supplementary FIGS. 2b-d). There was no change in signal line shape.
(iii) Unlike L-arg, other amino acids showed a change in mode chemical shift after particle formation, (see arrows in Supplementary FIG. 2). The magnitude depended on the peak. The largest shift for L-lys was at its peak at 3.90 ppm, after which particle formation migrated to 3.75. The L-leu peak at 3.88 ppm moved to 3.73, while the gly peak changed from 3.69 ppm to 3.55. In contrast, all L-arg peaks shifted downfield by less than 0.02 ppm.

The interactions between insulin and the selected amino acid is complex. Of the four amino acids studied, L-arg appears had the most interesting behaviour. Changes in both the insulin and L-arg signal were continuous with ZnCl₂ addition, as opposed to occurring only at particle formation. This may suggest the formation of multimers of protein prior to reaching the critical ZnCl₂ concentration. Due to the similarities between the molecular structure of L-arg and L-lys and the difference in the insulin behaviour, it is likely that the specific behaviour was linked to the amine-rich side chain.

The Effect of L-arg and ZnCl₂ Concentration on Insulin Core Particle Formation

There was a complex relationship between L-arg and ZnCl₂ dependent on solution pH. Particles did not form below pH 6.5 regardless of solution salinity, this was typically the case when the L-arg concentration was <2 mg/mL. When the pH was in the “goldilocks” zone, between ˜6.5 and ˜9.5, the ZnCl₂ concentration required to form optimised insulin particles and insulin-SiNPs depended upon the L-arg concentration. A measurable change in the DLS signal (indicative of insulin association) was observed with 0.1 mM ZnCl₂ when the L-arg concentration was 2 mg/mL, ˜0.3 when it was 3 mg/mL and 0.4 when it was 4 mg/mL (FIG. 2d). A corresponding change in the insulin signal was seen with addition of 0.2 mM ZnCl₂. Stable particle dispersions formed with ˜0.5 mM ZnCl₂ when L-arg was 2 mg/mL and ˜0.75 mM ZnCl₂ when it was 3 mg/mL. Furthermore, the insulin particles formed with 3 mg/mL L-arg (molar ratio: insulin to L-arg of 1 to 99) were twice as large as those made with 2 mg/mL (molar ratio of insulin to L-arg of 1 to 66) with a ˜250 nm diameter and ˜120 nm diameter, respectively (Supplementary FIG. 4 and Supplementary Table 1). In terms of morphology, the insulin-SiNPs made with ≤2 mg/mL L-arg had a clearer separation between the core and the shell than those made with 3 mg/mL (inserts in Supplementary FIG. 4).

Other Factors

Several additional factors were observed to affect insulin particle synthesis.

(i) with all other factors being equal, more ZnCl₂ was required for particle formation to occur if the HCl concentration used during Step 1 was low; (ii) Impurities, especially other salts, impacted upon the required ZnCl₂ concentration and stability of the final particles; (iii) the speed of the ZnCl₂ addition as well as homogenization may lead to the formation of regions with high local ion concentration resulting in larger insulin particle diameter and/or bimodal distributions, especially with synthesis scale up (discussed below).

Insulin-SiNP Synthesis Using an Insulin: L-Arg Mass Ratio of 1:2

While using an insulin:L-arg molar ratio of 1:99 was preferable for synthesis reproducibility, it was also possible to make particles using a ratio of 1:66 with no further alterations provided there was a fine control over the synthesis conditions. Indeed, insulin core particles and resulting insulin-SiNPs made this way are typically smaller: ˜220 nm and 260 nm in diameter, respectively (Supplementary FIG. 4). They also had a different morphology according to TEM where the core shell structure was more apparent. Furthermore, the loading efficiency of this formulation was observed to be 53% (N=2), consistent with that observed for typical insulin-SiNPs.

TEOS Additions

Several factors impacted the loading capacity, size and morphology of the final SiNP. Specifically: (i) pH and stirring, (ii) rate of addition, (iii) total volume of TEOS/mL of particles added and (iv) reaction time. The first two affect the rate of TEOS hydrolysis, which in turn influences monomer silicic acid monomer generation and also the crosslinking of insulin- and exenatide-SiNPs². We found that slow monomer generation was more beneficial to protein crosslinking, as it leads to more uniform particles with higher loading capacity and, in the case of insulin-SiNPs, a more definitive core shell morphology.

TEOS Hydrolysis Rationale

Silica forms in water spontaneously from TEOS in these conditions above a critical concentration². It would be energetically preferable for silicic acid molecules to remain in the particle. Thus, if silicic acid concentration is kept below a critical value, its molecules will preferentially crosslink protein particles rather than form a separate silica phase. Morphology and reaction rate were controlled by controlling TEOS hydrolysis at a constant volume/mL. Hydrolysis rate was controlled by the size of the TEOS/water interface, temperature and a basic pH of 10. The images above show how the unwashed sample changes with increasing TEOS hydrolysis (Supplementary FIG. 11). Appearance of a secondary silica phase and crosslinking was seen with an increased hydrolysis rate. Thus, slower TEOS hydrolysis is preferable.

Volume of TEOS Influences Particle Morphology

A different volume of TEOS was added to each of insulin, exenatide and BSA core particles. The dispersion was incubated for ˜24 and ˜48 h at ˜20° C. and the resulting particles were measured by DLS and TEM before and after purification. The insulin-SiNPs had a pronounced core-shell, whereas exenatide- and BSA-SiNPs were more uniform. Increasing the TEOS added to insulin particles from 0.5 μL per mL to 2 μL per mL increased particle size in a volume-independent manner over the volume range (Supplementary FIG. 11). However, particle morphology changed with TEOS concentration. An increase in TEOS volume/mL yielded an increase in the fraction of shell in the overall diameter: 0.5 μL/mL yielded a shell thickness of ˜10 nm or 9% of the particle diameter, 1 μL/mL yielded a shell thickness of ˜15 nm or 20% of the particle diameter, and 1.8 μL/mL yielded a shell thickness of ˜20 nm or 25% of the particle diameter (Supplementary FIGS. 12 and 13). Increasing TEOS volume to 2 μL and above led to loss of the core-shell structure. The reaction incubation time impacted upon particle size and morphology. An increase from 20 to 40 h lead to a larger insulin-SiNP final size and a discriminated core-shell. Increasing the TEOS volume had a volume-dependent effect on exenatide-SiNP size. A larger volume led to an increase in particle size: 1 μL of TEOS per mL of exenatide particles increased their diameter by 12 nm, 3 μL of TEOS per mL of exenatide particles led to a size increase of 86 nm, while 5 μL of TEOS per mL of exenatide particles increased their diameter by 116 nm (Supplementary FIG. 14). Reaction time was also important for exenatide-SiNPs, as they aggregated if more than 3 μL per mL TEOS was used (Supplementary FIG. 15).

Loading Capacity and Loading Efficiency

Loading Capacity

Several approaches were applied to measure the chemical composition of the SiNP. Both direct- and indirect measurements of the particle peptide content were attempted by HPLC, NMR, IR, elemental analysis, SDS-PAGE, CD, and protein fluorescence labelling. Since dispersing exenatide in water did not require HCl, initial methodology was established using Exenatide-SiNP.

Directly measuring the protein content of particles requires particle dissolution and complex sample preparation. Methodologies were separated in several categories based on best outcome requirements: (i) those for which protein structure is not essential for recognition (pH>10), i.e. NMR and HPLC; (ii) those for which at least the primary protein structure should be maintained: IR, SDS-PAGE, CD and micro BCA and (iii) methods which are not structure-sensitive: elemental analysis and protein fluorescent labelling. A detailed view of conditions for silica dissolution can be found^{3, 4}. Complete dissolution of the SiNP proved difficult, and as a result, IR, SDS-PAGE, CD and micro BCA were not applicable. Neither was it possible to measure the chemical composition using fluorescence labelling, NMR, HPLC, and elemental analysis. Of those, fluorescently-labelling the protein prior to particle formation may have unintended effects on the process and leading to possible changes in the composition of the final dispersion or the functionality of the protein. Furthermore, it introduces several additional steps which increases the time of each reaction and is not practical when used for insulin. Elemental analysis is destructive and requires >1 mg of sample. Both were used sparingly. On the other hand, NMR and HPLC, while destructive in this context, require less sample (≈0.5 mg), can be used in tandem, and were chosen for batch composition studies. Supernatant content could be measured by most of the above methods and was used to estimate loading efficiency.

Loading Efficiency

Measuring the peptide concentrations in the supernatants was possible using most methods except for SDS-PAGE and IR, where the signal-to-noise ratio was too low, and for elemental analysis where there were high sample requirements. The capacity of NMR to simultaneously and quantitatively measure distinct species in native conditions makes it a powerful characterisation tool. Similar to direct measurement, fluorescence labelling of the protein allows for a rapid measurement of the loading efficiency, but the impracticality of the additional steps and possible influence on the reaction made it inconvenient for frequent use. CD and BCA were straightforward to use, but were problematic in distinguishing between insulin and L-arg, as both would be detectable.

Reproducibility

Synthesis Reproducibility

Synthesis reproducibility of insulin-entrapped particles in terms of physicochemical properties was measured in three ways: by comparing five side-by-side batches by a single researcher made in a single study, using a side-by-side comparison of batches synthesised by two researchers, and by an evaluation of batches made by the same researcher using the same protocol. Studying the variability between five scientific replicates made side-by-side on the same day was used to estimate variability in the synthesis. This variability depended on the stage of the synthesis: insulin core particle batches made on the same day had a Z average hydrodynamic diameter of 329 nm with a variability of 11% (37 nm). This was above the measurement variability (typically ˜10 nm or 3%). The same particles had a number mean hydrodynamic diameter of 236 nm with a variability of 11% (Supplementary FIGS. 22 and 23, and Supplementary Table 3). The variability in mean sizes between unwashed insulin-SiNPs was ˜28%, which was attributed to the mixture of species in the dispersion at this stage. When washed, insulin-SiNP batch-to-batch variability reduced to 11% or 48 nm for ˜455 nm particles. However, while the measured difference in mean diameter was ˜20 nm, the peak width of the distribution was ˜350 nm. Thus, our data suggests that while there was measurable difference between the mean sizes of five scientific repeats, it was relatively small compared to particle size and distribution width. This is evident from TEM images (Supplementary FIG. 24) where 100-300 nm particles can be seen. In all cases the particles appear as discrete oblongs with relatively irregular shape. There was a mix of sizes, further confirming the DLS results. None of the batches had a pronounced core-shell, which might be attributable to the surface area change in these sample due to the relatively large diameter of the initial insulin particles (˜330 nm) compared to the average (˜290 nm).

Examining variability of the synthesis was further assessed. The average hydrodynamic diameter measured for insulin core particles was 290 nm, with a relative SD of 18% (N=34). For the insulin-SiNPs, it was 488 nm with a variability of 38% (N=35), and for washed insulin-SiNPs, it was 451 nm with a variability of 15% (N=24) (Supplementary FIG. 25, Supplementary FIG. 4). Applying the same analysis as described above, the variability in mean diameter of 53 nm for insulin particles was relatively significant compared to a peak width of ˜300 nm. While it is not expected that variability on this scale would result in significant changes in particle properties it may affect morphology and loading capacity. Furthermore the 53 nm measured here are more than twice of that estimated in the scientific repeats experiment (20 nm). This was expected due to the much higher N number and lower control over conditions. We expect that the batch-to-batch variability will be greatly decreased if a more automated approach is adopted, with use of well controlled homogenization and accurate pumps. It is also recommended that ZnCl₂ solutions are standardised by conductivity rather than calculated ZnCl₂ concentration, as even small changes can impact on particle size. Measuring the variability in the synthesis from two researchers estimates the variability in the synthesis between two independent operators with minimum communication and training. Operator 1 synthesised three samples with an average diameter of 256 nm and a relative standard deviation of 4%, similar to the variability between individual DLS measurements of the same sample. Operator 2 synthesised two samples with an average diameter of 272 nm and a relative standard deviation of 8%, slightly larger than instrumental error (Supplementary FIG. 26 and Supplementary Table 5). Overall, the average size of all samples made by operator 1 and 2 was 262 nm±16 nm (6%); this variability is within that seen when comparing scientific repeats.

A similar side-by-side experiment was used to establish the reproducibility of particles. The average hydrodynamic diameter of five scientific repeats was 126 nm±14 nm (11%) for exenatide core particles and 133±13 nm (9%) for washed exenatide-SiNPs (Supplementary FIG. 27 and Supplementary Table 6). A similar analysis can be made in terms of particle composition. In a side-by-side experiment, loading capacity of insulin-SiNP samples was found to average 63%±10% insulin by NMR and 62%±17% by HPLC (Supplementary Table 7). Expanding the sample selection to all measured samples, synthesised by the optimised procedure, changes the average insulin content to 59%±11% by NMR (N=18) and 57%±18% by HPLC (N=13). Further studies using elemental analysis yielded a loading capacity of 51%±10% (N=2).

Characterisation Reproducibility

We compared the physicochemical properties of an exenatide-SiNP batch, divided between UCD and Sanofi (Montpellier). Particles were initially synthesised and characterised at UCD, and the remainder of the batch was shipped to Montpellier in a liquid suspension. Their capacity to reduce plasma glucose was tested using a glucose challenge in a mouse model.

Comparing Particle Size

Exenatide-SiNPs were synthesised and characterised at UCD after which the liquid dispersion was sent to Sanofi for repeat analysis using the same protocol. Exenatide-SiNPs unwashed, and after one and two washing steps were studied. Size of the particles was measured through DLS, Z average, intensity and number mean. Variability between particle diameter between the two institutions was less than 5% in all cases (Supplementary Table 8). Particle size distribution obtained at both institutions were comparable (Supplementary FIG. 28). Variability in PDI was below 38% in all cases.

Comparing Zeta Potential

At both labs, zeta potential was measured using a Malvern Zetasizer ZS series with Zeta potential cuvettes (model number: DTS1070). Measurements at UCD were done in a 1/100 dilution of PBS (I=1.4×10⁻³ M), while those at Sanofi were done in deionised water. All samples had a negative zeta potential, however, values measured at Sanofi were closer to neutral (Supplementary Table 9). This can be attributed to the lower medium conductivity in Sanofi measurements. Zeta potential distributions between both institutions were comparable (Supplementary FIG. 29).

Comparing Exenatide-SiNP Loading Capacity

In both labs particles were dissolved in basic conditions and the exenatide content was measured using a C₁₈ column in reverse phase HPLC. The mobile phase used was 69% water, 30% acetonitrile and 1% THF. Results were compared to a calibration curve. Exenatide content for the exenatide-SiNPs after one and two washing cycles was less than 10% (Supplementary Table 10). This is similar to what was obtained between batches.

Further Considerations

Reaction Scale-Up

Though the reaction was found to be scalable, several factors were important for the quality of the final product. (i) Reaction volume homogeneity: i.e. the appearance of concentration gradients within the reaction leading to a broad or bi-modal distributed particle population. This problem was addressed by appropriate homogenization and reaction vessel choice; (ii) Regions with high local ZnCl₂ concentration:ZnCl₂ addition rate and homogenization were important at higher volumes. Thus, the addition rate was reduced and an appropriate homogenization strategy adopted; (iii) Regions with high local TEOS concentration: both the particle crosslinking/coating and the formation of a secondary silica phase were dependent upon the rate of TEOS hydrolysis, which relates to reaction conditions and TEOS concentration. In high volume reactions, local TEOS maxima can appear due to reagent point addition. Similar to the two previous considerations, the problem can be resolved with adequate homogenization and a reduction of the TEOS addition rate using microfluidization. No significant size variability was detected when these considerations were taken into account (Supplementary FIG. 30 and Supplementary Table 11).

Using NMR to Monitor the Reaction Mechanism

We further used NMR to monitor the insulin-SiNP synthesis, conducted in D₂O. There was little change in the L-arg and insulin NMR signals after they were mixed, indicating weak association (Supplementary FIG. 16b). With ZnCl₂ addition and consecutive particle formation, the insulin signal was lost, while the integration of L-arg signal was reduced by ˜9% and lost its fine structure. This suggests that most of the insulin formed aggregates with ZnCl₂ and L-arg (Supplementary FIG. 16b). Between 50-60% of the insulin signal reappeared following TEOS addition, while more of the fine structure of L-arg was lost (Supplementary FIG. 16b); this correlated well with the ˜50% loading efficiency (Supplementary FIG. 16c). It is probable that weakly-interacting insulin dissociates from the particle, while L-arg adsorbs on the SiNP surface. The ethanol released as a result of TEOS hydrolysis was consistent with the 2 μL per mL precursor added (Supplementary FIG. 16b). All weakly-associated contamination was removed after two washes (Supplementary FIG. 16b). Finally, insulin and L-arg were released when particles were dissolved in 0.2 M NaOH. Similar observations were made for exenatide-SiNP (Supplementary FIG. 17).

Long Term Sample Storage: Lyophilization

An insulin-SiNP batch was separated in seven aliquots, one was left in water, while 2, 4 and 6% of sucrose or trehalose were added to six aliquots. The resulting dispersions were further separated in two sets. One set (1^{st}) was left at room temperature (˜20° C.), while the other (2^{nd}) was frozen at −80° C. overnight and lyophilised over 36 h. Samples of the 2^{nd} set were reconstituted in water and dispersion was compared to their counterparts from the 1^{st} set. We found that even at the lowest concentration of either sugar, the dispersions of the 1^{st} and 2^{nd} set were similar, while samples aggregated in the absence of sugar (Supplementary FIGS. 31 and 32).

Materials and Methods

Materials

Methods

Insulin Core Particle Synthesis Using an Insulin:L-arg Molar Ratio of 1:99

Preparing the Insulin Suspension.

The required mass of insulin (insulin glulisine, Sanofi) was weighed in a clean, dry plastic container. HPLC grade water was added so that the final concentration was 2 mg/mL. Insulin did not dissolve, but formed a white. 10 μL/mL 1 M HCl was added to the heterogeneous mixture which was further vortexed. A transparent colourless solution was obtained, with no floating particles.

Core Particle Synthesis.

6 mg/mL L-arg was added to the insulin solution in a 1:1 volumetric ratio so that the final concentrations were 1 mg/mL insulin and 3 mg/mL L-arg. The solution was vortexed for a few seconds and left for several minutes to equilibrate. The pH of the solution was >8.5. This is a DLS point. ZnCl₂ was kept as a stock solution at a concentration of 200 mM in HPLC grade water following sonication. While the insulin+L-arg was equilibrating, the ZnCl₂ stock solution was diluted 20 fold in HPLC grade water to a final concentration of 10 mM. Sonication/vortexing is recommended to ensure the homogenization of stock and diluted solutions. 3×30 μL/mL aliquots of the diluted ZnCl₂ solution were added to the insulin+L-arg solution with homogenization between each addition. The solution was colourless and clear. Additional ZnCl₂ was added in 5 μL/mL increments with agitation until a white, turbid suspension was formed. In a typical synthesis, 70 μL ZnCl₂ was required to cause aggregation. The solution, at this stage was turbid but transparent when held up to light. This is a DLS point. The process was pH-dependent, as insulin did not aggregate if the pH range was not 8-10.

TEOS Addition and Particle Purification.

The core particles were left for at least 1 h to equilibrate before adding 1 μL/mL TEOS to the solution. During that time continual agitation was provided. If the reaction volume is >10 mL, slow spinning ensured homogeneity. If the reaction volume was high, TEOS was added in several steps to prevent generation of monomer in the solution and thereby a secondary silica phase. The suspension was left for at least 24-48 h. This is a DLS point. To wash, particles were distributed in 2 mL Eppendorf tubes and spun at 14 000 rpm for 30 min. The supernatant was removed and the particles were dispersed in water by gently pipetting them up and down in the Eppendrof, flicking with a finger and/or vortexing. This is a DLS point. Insulin-SiNPs were washed twice by centrifugation.

Insulin Core Particle Synthesis Using an Insulin:L-arg Molar Ratio of 1:66

Deionized water (HPLC-grade) was syringe-filtered through 0.2 μm filter membranes. An aliquot of this water was acidified to pH 2 with HCl. Concentrated L-arg (0.14 M) and ZnCl₂ (0.08 M) stock solutions were prepared. These solutions were sonicated for 10 min to ensure complete dissolution. Insulin was weighed out on a microbalance such that each sample tube contained 1 mg. L-arg and ZnCl₂ solutions were diluted to 0.023 M and 0.011 M respectively. Insulin was solubilized in HCl (0.01 M) and vortexed for 30 s (15 Hz). Insulin stock (500 μL) was added to an empty microcentrifuge at a concentration of 1 mg/mL and shaken at 25° C., 500 RPM. L-arg (500 μL) was then added to this solution (c=2 mg/mL) while shaking at 900 RPM. Subsequently, an aliquot of ZnCl₂ solution was added to the sample tube such that the final concentrations in the sample tube were:

| Reagent | Molar Concentration |
|---|---|
| Insulin | 0.17 mM |
| L-arg | 11.40 mM |
| ZnCl₂ | 0.55 mM |

Upon addition of ZnCl₂, a cloudy colloidal suspension was observed, which may be checked by shining a laser pointer through the sample tube. If colloids formed, a scattered beam was observed through the suspension. If no scattering occurred, peptide nanoparticles were unlikely to be present. If a colloidal suspension did not form, a further aliquot addition of ZnCl₂ may be added to the sample tube. Insulin core nanoparticles formed for molar ratios of ZnCl₂:L-arg from 1:21 down to 1:14. At low ZnCl₂ concentrations (molar ratios below 1:21), peptide nanoparticles formed, but hexamers were also likely present. Larger aggregates formed at high ZnCl₂ concentrations, thus, to improve reproducibility of the final peptide nanoparticle size, an optimal molar ratio of 1:21 was chosen which returned a mean hydrodynamic nanoparticle size of 220 nm. Samples were left shaking for 6 h after ZnCl₂ addition to ensure the sample had equilibrated. Samples were left overnight before TEOS addition. A small volume of TEOS (2 μL) is added (t=17 h) to each sample tube at 900 RPM; temp=37° C. Samples were left for 24 h to ensure complete hydrolysis of TEOS. Insulin-SiNP were washed using a microcentrifuge at 14000 RPM (16,873 g) for 15 min. The supernatant was carefully removed with a pipette and the pellet was re-suspended in deionized water.

Samples were checked using DLS at two stages: after preparation of peptide nanoparticles and after TEOS addition. An increase in the hydrodynamic size was observed after addition of TEOS indicating the formation of a silica shell on the outer peptide nanoparticle surface. After washing via centrifugation and resuspension, TEM samples were prepared by depositing <5 μL of suspension on a TEM grid. The sample was then covered and allowed to dry in ambient conditions. Peptide secondary structure conformation may be assessed at any stage of the process by depositing solution/suspension into a quartz cuvette and performing a circular dichroism measurement using a spectropolarimeter (180-300 nm).

Exenatide-SiNP Synthesis

Exenatide was solvated in HPLC grade water at a concentration of 3 mg/mL. The mixture was homogenized using inversion and vortexing for 30 s and left to equilibrate for 1 min. 166 μL/mL of 24 or 45 mg/mL L-arg was added, depending on required final size. The former results in exenatide core particles with a mean dimeter of 50-100 nm, while the latter yields ˜200 nm ones. The solution was homogenized again using vortexing and left for further 5 min to equilibrate. The final solution was a clear, colourless liquid with a pH of around 9. 100 mM ZnCl₂ was added slowly to the exenatide+L-arg solution using 9×10 μL/mL aliquots once every 10 min. The resulting exenatide+L-arg+ZnCl₂ solution was a clear, colourless liquid. It was left stationary or, if the volume of the reaction was large, slowly spinning at 20-25° C. for 24 or 48 h, depending on particle size for the particle to grow.

TEOS (2 μL/mL) was added to the synthesized exenatide core particles. The reaction was left either stationary or slowly spinning for further 24-48 h. This is a DLS point. To wash, particles were distributed in 2 mL Eppendorf tubes and spun at 14 000 rpm for 30 min. The supernatant was removed and the particles were redisposed in water by gently pipetting them up and down in the Eppendorf, flicking with a finger and/or vortexing. This is a DLS point. The final suspension was a semi-turbid, white liquid. The final particle size was characterised as for insulin-SiNP. Typical exenatide core particle and washed exenatide-SiNPs had a Z-Average between 100-200 nm with a PdI<0.2. Encapsulation efficiency was >90% (by NMR) and ≈50% (by NMR and HPLC), respectively. Exenatide core particles had a loading capacity of ≈50% (by NMR, HPLC and EA).

BSA-SiNP Synthesis

BSA was solvated in HPLC grade water at a concentration of 3.1 mg/mL. The mixture was homogenized using inversion and vortexing for 30 s and left to equilibrate for a further minute. 250 μL/mL of 24 mg/mL. L-arg was then added and the solution homogenized again using vortexing and left for further 5 min to equilibrate. The final solution was a clear, colourless liquid with a pH of around 9. 200 mM ZnCl₂ was added slowly to the BSA+L-arg solution as 2×20 μL/mL and 1×5 μL/mL aliquots once every 10 min. The time between additions was measured using DLS. The mixed solution was a clear, colourless liquid and appeared largely unchanged. It was left stationary or, if the volume of the reaction was large, slowly spinning at 20-25° C. for 4 hours for the particle to grow. TEOS was added in a volume of 2 μL/mL to the core particles. The reaction was left slowly spinning for further 72 h. To wash, the particles were distributed in 2 mL Eppendorf tubes and spun at 14 000 rpm for 30 min. The supernatant was removed as described as above. The final particle size was characterised as for insulin-SiNP.

Storage

All particles were stored at 4° C. for up to a month.

Characterisation Methods

DLS

The volume of solution was >0.5 mL in water or aqueous buffer all cases.

Proteins: Proteins were dispersed in water or appropriate media at a concentration of ≥1 mg/mL.

Protein core particles: The pure protein particles were carefully transferred into the cuvette without further dilution.

SiNP, unwashed or washed: These particles were either measured as synthesized or diluted 1/100 in the appropriate media.

TEM

Particles (˜1 mg/mL) were diluted 1/50 in water or an appropriate buffer. 5-10 μL of the vortexed suspension were transferred to a 400 mesh copper grid and left to dry for >1 h. A standard Techni 12 instrument set to 120 keV was used in all cases. TEM data was analysed manually using ImageJ®.

Circular Dichroism

A Jasco J-810 was used for all measurements. Parameters used were as follows:
Measurement window: 200-300 nm.
Data pitch: 0.5 nm
Band width: 2 nm
Response: 4 sec
Scanning speed 50 nm/min
Number of runs in measurement (accumulation): 8
Temperature: Room temperature (20° C.)

Insulin, L-arg solutions, mixtures and insulin core and SiNPs were measured at appropriate concentration in an ultra-low volume (1=1 mm) cuvette and at appropriate concentrations. Comparison was made using the intensity of the insulin peak at 220 nm and the L-arg peak at 210 nm. Results were compared to standard curves. The high tension (HT) in all cases was below 800 V unless otherwise specified.

HPLC

A C18 column and a 69% water, 30% acetonitrile and 1% THF mobile phase were optimal for measuring both free exenatide and insulin, as well as peptides released from particles. Solutions used had a pH<10.

Measuring Free Protein.

Water was run first for 20 min at a rate of 0.5 mL/min to let the column equilibrate with the mobile phase and then again for 10 min at a rate of 1 mg/mL. Samples were run at 1 mg/mL flow rate in the above mentioned conditions.

Measuring the Peptide/Protein Loading Capacity of SiNPs.

Typically, particles were dissolved using 0.2 M NaOH for >3 h at 37° C. Then the pH was adjusted to ˜8 using 1M HCl and the samples were transferred to a standard HPLC vial. Water was run as a sample for 30 minutes prior to sample measurement. Samples were run within 2 h of the pH adjustment to prevent silica reconstitution. The final result was compared to a standard curve made of the protein under study at the same conditions. Typically it takes the sample<10 min to elute out of the column at a flow of 1 mL/min.

Calibration Curve for Estimating the Loading Capacity of Protein-SiNPs.

The protein was treated as a sample. It was dispersed in a predetermined concentration and an amount of HCl (10 μL/mL) was added in the case of Insulin. NaOH was added so that its final concentration was 0.2 M. The proteins were left for at least 3 h at 37° C. After which the pH was brought down <10 using HCl, as measured by pH paper. The as obtained protein solution was diluted and measured the same way as described above.

NMR

Preparing a Calibration Curve

Protein concentration was measured through a calibration curve made in appropriate conditions. Calibration for free protein was carried out either by dissolving it in D₂O or, in the case of insulin, D₂O/HCl (pH 2), after which the pH was fixed to 7 in all cases. Calibration for determining protein concentration after dissolution was made by incubating the free protein in 0.2 M NaOH at 37° C. for several hours and measuring the solution without further pH adjustments.

Measuring Unwashed- or Washed Particle Dispersions.

Insulin-L-arg mixtures, protein core particles or protein-SiNPs were either made in D₂O or spun into D₂O twice without any intentional dilution.

Measuring Loading Capacity.

Typically a known sample volume was dried in a pre-weighed Eppendorf tube (using a 6 place balance). The mass of the dry nanoparticles was measured and subsequently dissolved in 1 mL of 0.2 M NaOH, 1 mM DSS solution for >5 h at 37° C.

NMR Settings.

The peak at 0 ppm of (4,4-dimethyl-4-silapentane-1-sulfonic acid) at a concentration of 1 mM was used as an internal standard for all measurements. They were done with 16 scans with a relaxation time of 25 sec in a Varian 400 and 600 MHz instrument using 600 MHz NMR tubes.

Data Analysis.

Data was analysed using the automatic and manual peak fitting in the MestReNova V11 software.

In Vivo Methods

Rat Intestinal Instillations

Male Wistar rats (UCD Biomedical Facility and Charles River, UK, 7-10 weeks of age, bodyweight 300-400 g) were housed under environmentally-controlled conditions of humidity and a 12:12 h light/dark cycle. Animals had access to filtered water and standard laboratory chow ad lib. All experiments were conducted in accordance with Health Products Regulatory Agency project authorisation AE18982/P036 and UCD Animal Research Ethics Committee protocol number 13-40. All procedures were performed under anaesthesia. Before anaesthesia, animals were fasted overnight with free access to filtered water. Isoflurane (Iso-Vet, 1000 mg/g isoflurane liquid for inhalation, Piramal Healthcare, UK) was used for induction (5 L/min mixed with 4 L/min O₂) and maintenance of anaesthesia (2 L/min mixed with 2 L/min O₂). The in vivo bioactivity of released insulin was investigated. Insulin solution or insulin released from SiNPs was injected (s.c) at 1 IU/kg using a 26 G needle. PBS was also administered as a control. In situ instillations were performed as previously described⁵ with minor modifications. In brief, midline laparotomy was performed and an intestinal loop (4-6 cm) was created using size 4 braided silk suture (Mersilk®, Ethicon Ltd., Uk). Care was taken to avoid obstructing any vasculature. The abdominal cavity was closed using a skin stapler (AZ Manipler 35W, Braun). PBS (control) or treatments (Insulin, insulin-SiNP (50 IU/kg insulin) were instilled using a 30 G needle. In the case of the insulin-SiNP, an equal volume of PBS was administered into the loop prior to the NP solution to aid dispersion. Glucose levels were measured with blood obtained from the tail vein every 20 min (T0-T120) using a glucometer (Accu-chek Aviva, Roche). Animals were euthanised by intracardiac injection of 0.5 mL of pentobarbital sodium (EUTHATAL™, 200 mg/ml, Merial Animal Health Ltd., UK) using a 21 G needle. After euthanasia, the intestinal loop was placed in 10% formalin and subsequently embedded in paraffin wax. 5 μm tissue sections were cut on a microtome (Leitz 1512; GMI, USA), mounted on adhesive coated slides, stained with haematoxylin/eosin (H&E) and Alcian blue and examined under light microscopy (Nikon Labphoto; Nikon, Japan).

Oral Administration of Exenatide-SiNPs to Mice

Female C57BL/6 mice with a body weight of 19-25 g were used. Mice were randomly assigned to treatment groups and deprived of food for 6 h prior to study start but with tap water ad libitum. Controls were treated orally with 0.1M TRIS buffer of pH 8.0 (n=12). Test groups were treated with exenatide-SiNPs (p. o., n=10) or exenatide (s.c., n=10) respective doses of 1 mg/kg and 10 μg/kg, respectively. An oral glucose tolerance test (OGTT) using 2 g/kg p.o. glucose was initiated 0.5 h after treatment. Blood glucose was determined between 0 h (=time point of drug treatment) and 4 h. The PK of exenatide was analysed 0.5 and 1 h after administration from K 3-EDTA-treated plasma using an exenatide ELISA (LLoQ=11.2 pg/mL).

### SUPPLEMENTARY REFERENCES

1. Lin, W., Insley, T., Tuttle, M.D., Zhu, L., Berthold, D. A., Král, P., et al. Control of protein orientation on gold nanoparticles. J. Phys. Chem. C. Nanomater. Interfaces 119, 21035-21043 (2015).
2. Hristov, D. R., Mahon, E. & Dawson, K. A. Controlling aqueous silica nanoparticle synthesis in the 10-100 nm range. Chemical Communications 51, 17420-17423 (2015).
3. Mahon, E., Hristov, D. R. & Dawson, K. A. Stabilising fluorescent silica nanoparticles against dissolution effects for biological studies. Chemical Communications 48, 7970-7972 (2012).
4. Meder, F. Thomas, S. S., Fitzpatrick, L. W., Alahmari, A., Wang, S., Beirne, J. G., et al. Labeling the structural integrity of nanoparticles for advanced in situ tracking in bionanotechnology. ACS Nano 10, 4660-4671 (2016).
5. Aguirre, T. A. S., Aversa, V., Rosa, M., Guterres, S. S., Pohlmann, A. R, Coulter, I., Brayden, D. J. Coated minispheres of salmon calcitonin target rat intestinal regions to achieve systemic bioavailability: Comparison between intestinal instillation and oral gavage. J Control Release 238, 242-252 (2016).

1. A method of preparing a silica-protein sub-micron particle, the method comprising contacting, in an aqueous medium having a pH in the range of about 7.5 to 10 and having an ionic strength in the range of about 1.2 to 60 mM,
(i) protein sub-micron particle cores comprising 0.2 to 3 μmoles, per mg of the protein sub-micron particle cores, of a basic compound of formula (I) or an ester and/or amide thereof 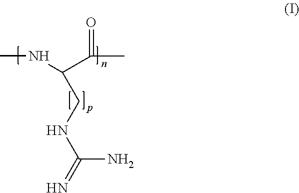
wherein n is an integer selected from 1 to 10, and p is an integer selected from 1 to 3,
with
(ii) orthosilicic acid or an ester thereof, in an amount equivalent to 5 to 60% (w/w) orthosilicic acid per mg of the protein sub-micron particle cores, to form the silica-protein sub-micron particle.

2. The method of claim 1, wherein the protein sub-micron particle cores comprising the basic compound of formula (I) are formed by
(a) dispersing 0.5 to 5.5 mg/mL of the protein in an aqueous medium having a pH of about 7.5 to 10 and having an ionic strength in the range of about 1.2 to 60 mM; and
(b) contacting the product of step (a) with the basic compound of formula (I) under reaction conditions to form the protein sub-micron particles comprising the basic compound of formula (I) in an amount equivalent to 0.2 to 2 mmoles of the basic compound of formula (I) per mg of sub-micron particles.

3. The method of claim 1, wherein the protein sub-micron particle cores comprising the basic compound of formula (I) are formed by
(c) dispersing 0.5 to 5.5 mg/mL of the protein and the basic compound of formula (I) in an aqueous medium having a pH of about 7.5 to 10 and having an ionic strength in the range of about 1.2 to 60 mM to form the protein sub-micron particle comprising the basic compound of formula (I) in an amount equivalent to 0.2 to 2 mmoles of arginine per mg of sub-micron particles.

4. The method of any one of claims 1-3, wherein the basic compound of formula (I) is L or D arginine, or an ester and/or amide thereof.

5. The method of any one of claims 1-3, the basic compound of formula (I) is a poly arginine (L or D), or an ester and/or amide thereof.

6. The method of claim 5, wherein the poly arginine is a dimer or a trimer.

7. The method of any one of claims 1-6, wherein the orthosilicic acid or the ester thereof is orthosilicic acid.

8. The method of any one of claims 1-6, wherein the orthosilicic acid or the ester thereof is an orthosilicate ester.

9. The method of claim 8, wherein the silicate ester retains 0 to 3 hydroxyl groups of the 4 hydroxyl groups of orthosilicic acid.

10. The method of claim 8, wherein each hydroxyl group of orthosilicic acid is independently esterified with C₁₋₅ alkyl; optionally, ethyl.

11. The method of claim 10, wherein each hydroxyl group is esterified with ethyl.

12. The method of any one of claims 1-11, wherein the orthosilicic acid or the ester thereof, is present in an amount equivalent to about 10 to 50% (w/w) orthosilicic acid per mg of protein sub-micron particles; wherein, optionally, when the protein is insulin, about 10 to 20% (w/w) orthosilicic acid per mg of protein sub-micron particles is present; and, when the protein is exenatide, about 10 to 50% (w/w) orthosilicic acid per mg of protein sub-micron particles is present.

13. The method of any one of claims 2 and 4-12, wherein, in step (a), about 0.85 to 4.00 mg/mL of the protein is dispersed; wherein, optionally, when the protein is insulin, about 0.85 to 1.5 mg/mL of the protein is dispersed; and, when the protein is exenatide, about 1.0 to 4.00 mg/mL of the protein is dispersed.

14. The method of any one of claims 3-13, wherein, in step (c), about 0.85 to 4.00 mg/mL of the protein is dispersed; wherein, optionally, when the protein is insulin, about 0.85 to 1.5 mg/mL of the protein is dispersed; and, when the protein is exenatide, about 1.0 to 4.0 mg/mL of the protein is dispersed.

15. The method of any one of claims 1-14, wherein the pH of the aqueous medium is in the range of about 8.9 to 9.7.

16. The method of any one of claims 1-15, wherein the ionic strength of the aqueous medium is in the range of about 2 to 35 mM.

17. The method of any one of claims 1-16, wherein the contacting step is carried out a temperature in the range of about 18 to 40° C.

18. The method of any one of claims 1 to 17, further comprising the step of separating the silica-protein sub-micron particle from the aqueous medium by centrifugation.

19. The method of any one of claims 1-18, wherein the silica-protein sub-micron particle has a diameter of between about 30 and 600 nm; optionally between about 50 and 300 nm; further optionally between about 80 and 300 nm.

20. The method of claim 19, wherein the protein is insulin and the silica-protein sub-micron particle has a diameter of between about 80 and 300 nm.

21. The method of claim 19, wherein the protein is exenatide and the silica-protein sub-micron particle has a diameter of between about 30 and 600 nm.

22. A method of making a GLP-1 receptor agonist sub-micron particle core, the method comprising the steps of:
a. contacting a peptide in an aqueous medium at a pH of about 5.0-8.0 and comprising about 0.3-1.3 mM peptide, wherein the peptide is a GLP-1 receptor agonist, with a basic compound of formula (I) or an ester and/or amide thereof: 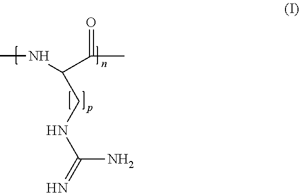
wherein n is an integer selected from 1 to 10, and p is an integer selected from 1 to 3;
in a molar ratio between the peptide and the basic compound of from 1:about 20 to 1:about 90 to form a step a. reaction mixture having a pH of about 7.5 to 10.0; optionally about 9.5;
b. contacting the step a. reaction mixture with zinc to form a step b. reaction mixture, wherein the ratio of peptide:basic compound:zinc in the step b. reaction mixture is 1:about 20 to 110:9 to 30, optionally between 1:48:16 to 22 and 1:90:20 to 25;
whereby the GLP-1 receptor agonist sub-micron particle core is formed.

23. The method of claim 22, wherein the GLP-1 receptor agonist is selected from exendin-4, exenatide (Byetta, Bydureon), liraglutide (Victoza), lixisenatide (Lyxumia), dulaglutide (Trulicity) albiglutide (Tanzeum) sitagliptin (Januvia, Janumet, Janumet XR, Juvisync), saxagliptin (Onglyza, Kombiglyze XR), alogliptin (Nesina, Kazano, Oseni), semaglutide (Ozempic) and linagliptin (Tradjenta, Jentadueto); optionally is exenatide or analogues thereof.

24. The method of claim 22 or 23, wherein the basic compound is arginine and the peptide is exenatide; and the concentration, in the step b. reaction mixture, of Zn is between about 6.5 and about 8.5 mM when the arginine is between about 25 and about 55 mM.

25. The method of any one of claims 22 to 24, wherein the amount of GLP-1 receptor agonist, in the protein sub-micron particle core, is between about 20% w/w and about 80% w/w; optionally between about 30% w/w and about 70% w/w; further optionally between about 35% w/w and about 60% w/w of the protein sub-micron particle core.

26. A method of making an insulin sub-micron particle core, the method comprising the steps of:
a. contacting insulin in an aqueous medium having a pH of less than about 6.0 and comprising about 0.50 to about 1.50 mg/mL insulin,
with a basic compound of formula (I) or an ester and/or amide thereof: 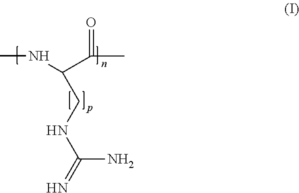
wherein n is an integer selected from 1 to 10, and p is an integer selected from 1 to 3;
in a molar ratio between the insulin and the basic compound of from about 1:40 to about 1:140 to form a step a. reaction mixture having a pH of about 7.5 to 10, optionally about 8.0 to 10.0;
b. contacting the step a. reaction mixture with zinc to form a step b. reaction mixture, wherein the molar ratio of insulin : zinc in the step b. reaction mixture is 1:about 0.5 to about 10;
wherein, optionally, the molar ratio of insulin:basic compound:zinc in the step b. reaction mixture is 1:about 66 to about 100:about 3 to about 4.5;
whereby the insulin sub-micron particle core is formed.

27. The method of claim 26, wherein the insulin in the aqueous medium comprises about 0.9-about 1.2 mg/mL insulin, optionally about 1 mg/mL insulin.

28. The method of claim 26 or 27, wherein n is 1 and p is 3 whereby the basic compound is arginine; and
the concentration, in the step b. reaction mixture, of Zn is about 0.35-0.65 mM, optionally about 0.5 mM, when the arginine concentration is 2 mg/mL; or
the concentration, in the step b. reaction mixture, of Zn is about 0.6-0.9 mM, optionally about 0.75 mM, when the arginine concentration is about 3 mg/mL.

29. The method of any one of claims 26-28, wherein the amount of insulin, in the protein sub-micron particle core, is between about 35% w/w and about 95% w/w; optionally between about 45% w/w and about 90% w/w; further optionally between about 50% w/w and about 75% w/w of the protein sub-micron particle core.

30. The method of any one of claims 26-29, wherein the insulin is a human insulin or an analog thereof; optionally selected from Lispro (Eli Lilly), Aspart (Novo Nordisk), Glulisine (Sanofi-Aventis), Detemir insulin (Novo Nordisk), Degludec insulin (Novo Nordisk), Glargine insulin (Sanofi-Aventis).

31. The method of any one of claims 26-30, wherein the aqueous medium has a pH of less than about 4.0, optionally about pH 2.5.

32. The method of any one of claims 22 to 31, wherein the basic compound is L-arginine.

33. The method of any one of claims 22-32, wherein the core comprises the peptide, the basic compound and the zinc.

34. The method of any one of claims 22-33, wherein the core consists of the peptide, the basic compound and the zinc.

35. The method of any one of claims 22-34, the method further comprising contacting, in an aqueous medium having a pH in the range of about 7.5 to 10 and having an ionic strength in the range of about 1.2 to 60 mM,
(i) protein sub-micron particle cores comprising 0.2 to 3 μmoles, per mg of the protein sub-micron particle cores, of a basic compound of formula (I) or an ester and/or amide thereof 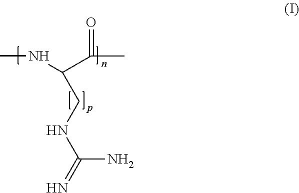
wherein n is an integer selected from 1 to 10, and p is an integer selected from 1 to 3,
with
(ii) orthosilicic acid or an ester thereof, in an amount equivalent to 5 to 60% (w/w) orthosilicic acid per mg of the protein sub-micron particle cores, to form the silica-protein sub-micron particle.

36. The method of any one of claims 22 to 34, further comprising providing an enteric coating on the protein sub-micron particle cores.

37. The method of any one of claims 22 to 34, the method comprising contacting, in an aqueous medium having a pH in the range of about 7.5 to 10 and having an ionic strength in the range of about 1.2 to 60 mM, the protein sub-micron particle cores prepared in accordance with any one of claims 22 to 37 with orthosilicic acid or an ester thereof, in an amount equivalent to 5 to 60% (w/w) orthosilicic acid per mg of the protein sub-micron particle cores, to form the silica-protein sub-micron particle.

38. The method of claim 35 or 37, wherein the protein sub-micron particle cores comprising the basic compound of formula (I) are formed by
(a) dispersing 0.5 to 5.5 mg/mL of the protein in an aqueous medium having a pH of about 7.5 to 10 and having an ionic strength in the range of about 1.2 to 60 mM; and
(b) contacting the product of step (a) with the basic compound of formula (I) under reaction conditions to form the protein sub-micron particles comprising the basic compound of formula (I) in an amount equivalent to 0.2 to 2 mmoles of the basic compound of formula (I) per mg of sub-micron particles.

39. The method of an one of claim 35, 37 or 38, wherein the protein sub-micron particle cores comprising the basic compound of formula (I) are formed by
(c) dispersing 0.5 to 5.5 mg/mL of the protein and the basic compound of formula (I) in an aqueous medium having a pH of about 7.5 to 10 and having an ionic strength in the range of about 1.2 to 60 mM to form the protein sub-micron particle comprising the basic compound of formula (I) in an amount equivalent to 0.2 to 2 mmoles of arginine per mg of sub-micron particles.

40. A silica-protein sub-micron particle formed by the methods of any one of claim 14-16, 38 or 39 for use in the treatment of diabetes.

41. The silica-protein sub-micron particle for use of claim 40, wherein the protein is insulin and the diabetes is Type I diabetes.

42. The silica-protein sub-micron particle for use of claim 40 or 41, wherein the diabetes is Type II diabetes.

43. An enteric coated -protein sub-micron particle formed by the method of claim 36 for use in the treatment of diabetes.

44. The enteric coated protein sub-micron particle for use of claim 43, wherein the protein is insulin and the diabetes is Type I diabetes.

45. The enteric coated sub-micron particle for use of claim 43 or 44, wherein the diabetes is Type II diabetes.
